(19) United States
(12) Patent Application Publication
(10) Pub. No.: US 2003/0148334 A1
(43) Pub. Date: Aug. 7, 2003

(54) Differentially-expressed genes and polypeptides in angiogenesis

(76) Inventors: Zairen Sun, Rockville, MD (US); Gilbert Jay, North Bethesda, MD (US)

Correspondence Address: ORIGENE TECHNOLOGIES, INCORPORATED, 6 TAFT COURT, SUITE 100, ROCKVILLE, MD, 20850, US

(21) Appl. No.: 10/268,994

(22) Filed: Oct. 11, 2002

Related U.S. Application Data
(60) Provisional application No. 60/328,395, filed on Oct. 12, 2001.

(51) Int. Cl. C12Q 1/68; G01N 33/574; G06F 19/00; G01N 33/48; G01N 33/50

(52) U.S. Cl. 435/6; 435/7.23; 702/20

(57) ABSTRACT

The present invention relates to all facets of polynucleotides, the polypeptides they encode, antibodies and specific binding partners thereto, and their applications to research, diagnosis, drug discovery, therapy, clinical medicine, forensic science and medicine, etc. The polynucleotides are expressed during angiogenesis and are therefore useful in variety of ways, including, but not limited to, as molecular markers, as drug targets, and for detecting, diagnosing, staging, monitoring, prognosticating, preventing or treating, determining predisposition to, etc., diseases and conditions, such as abnormal, insufficient, excessive, etc., angiogenesis, such as inflammatory diseases, such as rheumatoid arthritis, osteoarthritis, asthma, pulmonary fibrosis, age-related macular degeneration (ARMD), diabetic retinopathy, macular degeneration, and retinopathy of prematurity (ROP), endometriosis, cancer, Coats' disease, peripheral retinal neovascularization, neovascular glaucoma, psoriasis, retrolental fibroplasias, angiofibroma, inflammation, etc

This application claims the benefit of U.S. Provisional Application No. 60/328,395, filed Oct. 12, 2002, which is hereby incorporated by reference in its entirety.

### DESCRIPTION OF THE INVENTION

The present invention relates to all facets of polynucleotides, the polypeptides they encode, antibodies and specific binding partners thereto, and their applications to research, diagnosis, drug discovery, therapy, clinical medicine, forensic science and medicine, etc. The polynucleotides are expressed during angiogenesis and are therefore useful in variety of ways, including, but not limited to, as molecular markers, as drug targets, and for detecting, diagnosing, staging, monitoring, prognosticating, preventing or treating, determining predisposition to, etc., diseases and conditions, especially relating to the vascular system. The identification of specific genes, and groups of genes, expressed in pathways physiologically relevant to angiogenesis permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-related methods, e.g., advertising, displaying, offering, selling, etc., such products for sale, commercial use, licensing, etc.

Angiogenesis, the process of blood vessel formation, is a key event in many physiological processes that underlie normal and diseased tissue function. During ontogeny, angiogenesis is necessary to establish to the network of blood vessels required for normal cell, tissue and organ development and maintenance. In the adult organism, the production of new blood vessels is needed for organ homeostasis, e.g., in the cycling of the female endometrium, for blood vessel maturation during wound healing, and other processes involved in the maintenance of organism integrity. It also is important in regenerative medicine, including, e.g., in promoting tissue repair, tissue engineering, and the growth of new tissues, inside and outside the body.

Not all angiogenesis is beneficial. Inappropriate and ectopic expression of angiogenesis can be deleterious to an organism. A number of pathological conditions are associated with the growth of extraneous blood vessels. These include, e.g., diabetic retinopathy, neovascular glaucoma, psoriasis, retrolental fibroplasias, angiofibroma, inflammation, etc. In addition, the increased blood supply associated with cancerous and neoplastic tissue, encourages growth, leading to rapid tumor enlargement and metastasis.

Because of the importance of angiogenesis in many physiological processes, its regulation has application in a vast arena of technologies and treatments. For instance, induction of neoangiogenesis has been used for the treatment of ischemic myocardial diseases, and other conditions (e.g., ischemic limb, stroke) produced by the lack of adequate blood supply. See, e.g., Rosengart et al., Circulation, 100(5):468-74, 1999. In growth new tissues from progenitor and stem cells, angiogenesis is one of the key processes necessary. Where vascularization is undesirable, such as for cancer and the mentioned pathological conditions, inhibition of angiogenesis has been used as a treatment therapy. See, e.g., U.S. Pat. No. 6,024,688 for treating neoplasms using angiogenesis inhibitors.

A number of different factors have been identified which stimulate angiogenesis, e.g., by activating normally quiescent endothelial cells, by acting as a chemoattractant to developing capillaries, by stimulating gene expression, etc. These factors include, e.g. fibroblast growth factors, such as FGF-1 and FGF-2, vascular endothelial growth factor (VEGF), platelet-derived endothelial cell growth factor (PD-ECGF), etc. Inhibition of angiogenesis has been achieved using drugs, such as TNP-470, monoclonal antibodies, antisense nucleic acids and proteins, such as angiostatin and endostatin. See, e.g., Battegay, J. Mol. Med., 73, 333-346 (1995); Hanahan et al., Cell, 86, 353-364 (1996); Folkman, N. Engl. J. Med., 333, 1757-1763 (1995).

Activity of a polynucleotide or gene in modulating or regulating angiogenesis can be determined according to any effective in vivo or in vitro methods. One useful model to study angiogenesis is based on the observation that, when a reconstituted basement membrane matrix, such as Matrigel®, supplemented with growth factor (e.g., FGF-1), is injected subcutaneously into a host animal, endothelial cells are recruited into the matrix, forming new blood vessels over a period of several days. See, e.g., Passaniti et al., Lab. Invest., 67:519-528, 1992. By sampling the extract at different times, angiogenesis can be temporally dissected, permitting the identification of genes involved in all stages of angiogenesis, including, e.g., migration of endothelial cells into the matrix, commitment of endothelial cells to angiogenesis pathway, cell elongation and formation of sac-like spaces, and establishment of functional capillaries comprising connected, and linear structures containing red blood cells. To stabilize the growth factor and/or slow its release from the matrix, the growth factor can be bound to heparin or another stabilizing agent. The matrix can also be periodically re-infused with growth factor to enhance and extend the angiogenic process.

Other useful systems for studying angiogenesis, include, e.g., neovascularization of tumor explants (e.g., U.S. Pat. Nos. 5,192,744; 6,024,688), chicken chorioallantoic membrane (CAM) assay (e.g., Taylor and Folkman, Nature, 297:307-312, 1982; Eliceiri et al., J. Cell Biol., 140, 1255-1263, 1998), bovine capillary endothelial (BCE) cell assay (e.g., U.S. Pat. No. 6,024,688; Polverini, P. J. et al., Methods Enzymol., 198: 440-450, 1991), migration assays, HUVEC (human umbilical cord vascular endothelial cell) growth inhibition assay (e.g., U.S. Pat. No. 6,060,449).

The present invention relates to polynucleotides, and the polypeptides they encode, which are related to angiogenesis and the vascular system. These polynucleotides were identified using a model system for angiogenesis. In this system, a Matrige l™plug implant comprising FGF-1 is implanted subcutaneously into a host mouse. The initial bolus of FGF attracts endothelial cells into the implant, but does not result in new blood vessel formation. After about 10-15 days, the implant is re-infused with FGF-1. The FGF-1 stimulates the endothelial cells already present in the implant, initiating the process of angiogenesis. Tissue samples, removed at different intervals, can be analyzed to determine their gene expression patterns.

In results reported here, samples of the Matrigel™plug were harvested immediately prior to the re-injection with FGF-1, and then 1, 8, and 24 hours later. These samples were analyzed for gene expression, and differentially-expressed genes were identified by several methods. The results are summarized in Tables 1 and 2. At least eight different expression patterns were observed. These were classified according to whether the genes were up-(U) or down-(D) regulated, and whether the expression of the differentially-regulated gene was transient (T) or sustained (S). The term “transient” indicates that the gene expression levels changed temporarily, and then returned to the basal level. “Sustained” indicates that the expression levels changed, and then remained relatively stable. The sample removed prior to the FGF-1 re-infusion was used to establish the basal levels of gene expression, prior to angiogenesis. The following patterns were observed:

U1S: Gene up-regulated at 1-hour, and remained up in the 8- and 24-hour assays.

U8S: Gene up-regulated at 8-hours, and remained up in 24-hour assay.

U1T: Gene up-regulated at 1-hour, but returned to basal level in the 8- and 24-hour assays.

U8T: Gene up-regulated at 8-hours, but returned to basal level in the 24-hour assay.

D1S: Gene down-regulated at 1-hour, and remained down in the 8- and 24-hour assays.

D8S: Gene down-regulated at 8-hours, and remained down in the 24-hour assay.

D1T: Gene down-regulated at 1-hour, but returned to basal level in the 8- and 24-assays.

D8T: Gene down-regulated at 8-hours, but returned to basal level in 24-hour assay

At the first time point (“0”), endothelial and other cells are present in the Matrigel™ plug, but angiogenesis has not begun. After 1 hour, the endothelial cells have been stimulated by FGF, and genes involved in angiogenesis have been activated. By 8 hours, the endothelial cells have organized into a rudimentary tubes, but are not yet functional. At the end of 24 hours, the tubes have become functional, and are filled with blood cells.

Table 1 is a summary of the genes differentially-regulated over the 24-hour time course. H indicates that the expression levels were relatively high, and L indicates levels were relatively low. Both the mouse gene and its human homolog are listed in the table. About 166 different genes (human and mouse homologs are counted as one gene) were analyzed. The nucleotide and amino acid sequences are publicly available (hereby incorporated by reference in their entirety) and can be obtained, e.g., by searching the accession numbers listed in Table 1.

Not all subjects in an animal population will display the same gene expression profile, even when they express same or similar phenotypes. For instance, a group of patients may all have a cancer in which angiogenesis has been initiated, but they may not have 100% identical patterns of angiogenic gene expression. There are a number of reasons for such differences, including, e.g., variability among patient genetic backgrounds, differences in their exposure to environmental and other exogenous factors that influence gene expression, drug histories, cancer type, stage, and grade, allelic variations in the angiogenic genes, etc. For these reasons, there can be circumstances where one gene is inadequate to assess as a general tool to assess and treat angiogenesis. As a result, it may be desirable to use the genes in combination, rather than one at a time, to increase the diagnostic and therapeutic efficacy. While one particular gene may not be fully penetrant in all individuals exhibiting angiogenesis, using a set of genes enhances the probability of identifying angiogenesis is a broad population sample.

Table 2, for instance, shows that there are about 68 genes expressed immediately prior to angiogenesis, 67 genes during the first hour when the endothelial cells are stimulated by FGF, 97 genes during the formation of functional tubes, and about 79 genes when the tubes become functional and fill with blood. The expression of all, or subsets, can be useful to determine the extent and stage of angiogenesis, as well as devising therapeutic strategies. For instance, an assay for genes differentially-expressed during the initial stages of angiogenesis (i.e., 1 hour), can comprise all of the 67 genes identified in Table, 1, or subsets thereof. Selection of a subset can be based on any criteria, including, e.g., the genes which are expressed at the highest levels (e.g., Nos. 381 and 384), genes representative of one or more of the functional categories (e.g., nuclear regulatory factors, such as Nos. 324 and 348, or cell-surface proteins, such as No. 107), genes which show transient differential-expression (e.g., Nos. 135 and 307), genes which show sustained differential expression (e.g., Nos. 184 and 200), etc. Similarly, therapeutics, as discussed in more detail below, can target single genes or groups, e.g., cell-surface markers and/or cell-signaling molecules, genes expressed at high or low levels, etc.

As shown in Table 2, the genes can be sorted into different functional categories, e.g., categorized by function, signaling pathway, cellular compartment, components the gene and gene product interact with, etc., such as nuclear regulatory factors (NR) [e.g., transcription factors, RNA splicing, apoptosis]; extracellular matrix (ECM), including basement membrane and factors involved in ECM signaling [e.g., collagen, tensin]; cell-surface (CS) molecules; protein manufacture (PM) [e.g., translation factors, ER proteins, ribosomal factors, factors involved in secretion]; protein degradation (PD) [e.g., ubiquitin]; cell signaling (SI) [e.g., GTPases, ras and ras-like, cytokines, growth factors]; blood specific factors (BL); muscle (MU) [e.g., nebulin]; and endothelial cell factors [e.g., PAI]. These functional groups can be used alone, or in combination for diagnostic and therapeutic uses. For example, genes in the NR category which are up-regulated during angiogenesis can be used as an early target for therapeutic intervention. Genes products in CS category can be used in in vivo imaging or as therapeutic targets using antibodies.

Examples of genes in the various pathways, include, e.g., nuclear regulatory factors, e.g., zinc finger transcription factor, cytokine-nuclear factor n-pac, TACC2, topoisomerase, BCL2/E1b interacting protein, U1 small ribonucleoprotein 1 SNRP homolog, MCLI, beclin 1, bcl-2 related protein, Mcpr, nucleolar protein MSP58, TIA-1, ATP-dependent RNA hellicase, cyclin D3, proto-oncogene, disabled homolog 2, BACH1, HIP116, ETR103, BTB+CNC homolog, DNAJ portein, H1 histone, cdc5-related protein, AHNAK nucleoprotein, RD RNA binding protein, spastin, etc.; protein degradation pathways, e.g., ubiquitin fusion protein, proteasome, ubiqiutin proteases, etc.; protein manufacture pathways, e.g., ribophorin, ribosomal proteins, translation termination factor ETFI, p97, 28S, elongation factor, translation initiation factor, ribosomal L33-like, MRP-17, etc.; extracellular matrix, e.g., SPARC/osteonectin, pre-pro-collagen, tensin, proteoglycan, pro-collagen, cadherin 3, thrombospondin, connective tissue growth factor, pro-alpha collagen, nidogen, tensin, titin, etc.

Nucleic Acids

A mammalian polynucleotide, or fragment thereof, of the present invention is a polynucleotide having a nucleotide sequence obtainable from a natural source. When the species name is used, e.g., human, it indicates that the polynucleotide or polypeptide is obtainable from a natural source. It therefore includes naturally-occurring normal, naturally-occurring mutant, and naturally-occurring polymorphic alleles (e.g., SNPs), differentially-spliced transcripts, splice-variants, etc. By the term “naturally-occurring,” it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occurring mutations can include deletions (e.g., a truncated amino- or carboxy-terminus), substitutions, inversions, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, from a cDNA library, from a genomic library, etc. The polynucleotide can be obtained from a cell or tissue (e.g., from an embryonic or adult tissues) at a particular stage of development, having a desired genotype, phenotype, disease status, etc. The polynucleotides described in Table 1 can be partial sequences that correspond to full-length, naturally-occurring transcripts. The present invention includes, as well, full-length polynucleotides that comprise these partial sequences, e.g., genomic DNAs and polynucleotides comprising a start and stop codon, a start codon and a polyA tail, a transcription start and a polyA tail, etc. These sequences can be obtained by any suitable method, e.g., using a partial sequence as a probe to select a full-length cDNA from a library containing full-length inserts. A polynucleotide which “codes without interruption” refers to a polynucleotide having a continuous open reading frame (“ORF”) as compared to an ORF which is interrupted by introns or other noncoding sequences.

Polynucleotides and polypeptides (including any part of a differentially-expressed gene) can be excluded as compositions from the present invention if, e.g., listed in a publicly available databases on the day this application was filed and/or disclosed in a patent application having an earlier filing or priority date than this application and/or conceived and/or reduced to practice earlier than a polynucleotide in this application.

As described herein, the phrase “an isolated polynucleotide which is SEQ ID NO,” or “an isolated polynucleotide which is selected from SEQ ID NO,” refers to an isolated nucleic acid molecule from which the recited sequence was derived (e.g., a cDNA derived from mRNA; cDNA derived from genomic DNA). Because of sequencing errors, typographical errors, etc., the actual naturally-occurring sequence may differ from a SEQ ID listed herein. Thus, the phrase indicates the specific molecule from which the sequence was derived, rather than a molecule having that exact recited nucleotide sequence, analogously to how a culture depository number refers to a specific cloned fragment in a cryotube.

As explained in more detail below, a polynucleotide sequence of the invention can contain the complete sequence as shown in Table 1, degenerate sequences thereof, anti-sense, muteins thereof, genes comprising said sequences, full-length cDNAs comprising said sequences, complete genomic sequences, fragments thereof, homologs, primers, nucleic acid molecules which hybridize thereto, derivatives thereof, etc.

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention, or by searching nucleotide databases, such as GenBank and EMBL, for matches. Promoter and other regulatory regions (including both 5′ and 3′ regions, as well introns) can be identified upstream or downstream of coding and expressed RNAs, and assayed routinely for activity, e.g., by joining to a reporter gene (e.g., CAT, GFP, alkaline phosphatase, luciferase, galatosidase). A promoter obtained from a gene of the present invention can be used, e.g., in gene therapy to obtain tissue-specific expression of a heterologous gene (e.g., coding for a therapeutic product or cytotoxin). 5′ and 3′ sequences (including, UTRs and introns) can be used to modulate or regulate stability, transcription, and translation of nucleic acids, including the sequence to which is attached in nature, as well as heterologous nucleic acids.

Constructs

A polynucleotide of the present invention can comprise additional polynucleotide sequences, e.g., sequences to enhance expression, detection, uptake, cataloging, tagging, etc. A polynucleotide can include only coding sequence; a coding sequence and additional non-naturally occurring or heterologous coding sequence (e.g., sequences coding for leader, signal, secretory, targeting, enzymatic, fluorescent, antibiotic resistance, and other functional or diagnostic peptides); coding sequences and non-coding sequences, e.g., untranslated sequences at either a 5′ or 3′ end, or dispersed in the coding sequence, e.g., introns.

A polynucleotide according to the present invention also can comprise an expression control sequence operably linked to a polynucleotide as described above. The phrase “expression control sequence” means a polynucleotide sequence that regulates expression of a polypeptide coded for by a polynucleotide to which it is functionally (“operably”) linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can include an initiation codon and additional nucleotides to place a partial nucleotide sequence of the present invention in-frame in order to produce a polypeptide (e.g., pET vectors from Promega have been designed to permit a molecule to be inserted into all three reading frames to identify the one that results in polypeptide expression). Expression control sequences can be heterologous or endogenous to the normal gene.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR54 0, pRIT5 (Pharmacia). Eukaryotic: PWLNEO, pSV2CAT, pOG44, pXT1, pSG (Stratagene), pSVK3, PBPV, PMSG, pSVL (Pharmacia), pCR2.1/TOPO, pCRII/TOPO, pCR4/TOPO, pTrcHisB, pCMV6-XL4, etc. However, any other vector, e.g., plasmids, viruses, or parts thereof, may be used as long as they are replicable and viable in the desired host. The vector can also comprise sequences which enable it to replicate in the host whose genome is to be modified.

Hybridization

Polynucleotide hybridization, as discussed in more detail below, is useful in a variety of applications, including, in gene detection methods, for identifying mutations, for making mutations, to identify homologs in the same and different species, to identify related members of the same gene family, in diagnostic and prognostic assays, in therapeutic applications (e.g., where an antisense polynucleotide is used to inhibit expression), etc.

The ability of two single-stranded polynucleotide preparations to hybridize together is a measure of their nucleotide sequence complementarity, e.g., base-pairing between nucleotides, such as A-T, G-C, etc. The invention thus also relates to polynucleotides, and their complements, which hybridize to a polynucleotide comprising a nucleotide sequence as set forth in Table 1 and genomic sequences thereof. A nucleotide sequence hybridizing to the latter sequence will have a complementary polynucleotide strand, or act as a template for one in the presence of a polymerase (i.e., an appropriate polynucleotide synthesizing enzyme). The present invention includes both strands of polynucleotide, e.g., a sense strand and an anti-sense strand.

Hybridization conditions can be chosen to select polynucleotides which have a desired amount of nucleotide complementarity with the nucleotide sequences set forth in Table 1 and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complementarity, between the sequences. The present invention particularly relates to polynucleotide sequences which hybridize to the nucleotide sequences set forth in Table 1 or genomic sequences thereof, under low or high stringency conditions. These conditions can be used, e.g., to select corresponding homologs in non-human species.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 μg/ml denatured salmon sperm DNA, 5×Denhardt's solution, and 50% formamide), at 22-68° C., overnight, and then hybridized with a detectable polynucleotide probe under conditions appropriate to achieve the desired stringency. In general, when high homology or sequence identity is desired, a high temperature can be used (e.g., 65° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formamide can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 10% mismatch, less than 5% mismatch, etc., reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6 log₁₀[Na⁺]+0.41(% GC)−600/N where [Na⁺] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 90%, 95%, or 97%, nucleotide complementarity between the probe (e.g., a short polynucleotide of Table 1 or genomic sequences thereof) and a target polynucleotide.

Other homologs of polynucleotides of the present invention can be obtained from mammalian and non-mammalian sources according to various methods. For example, hybridization with a polynucleotide can be employed to select homologs, e.g., as described in Sambrook et al., Molecular Cloning, Chapter 11, 1989. Such homologs can have varying amounts of nucleotide and amino acid sequence identity and similarity to such polynucleotides of the present invention. Mammalian organisms include, e.g., mice, rats, monkeys, pigs, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc. The degree of nucleotide sequence identity between human and mouse can be about, e.g. 70% or more, 85% or more for open reading frames, etc.

Alignment

Alignments can be accomplished by using any effective algorithm. For pairwise alignments of DNA sequences, the methods described by Wilbur-Lipman (e.g., Wilbur and Lipman, Proc. Natl. Acad. Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent. Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various commercial and free sources of alignment programs are available, e.g., MegAlign by DNA Star, BLAST (National Center for Biotechnology Information), BCM (Baylor College of Medicine) Launcher, etc. BLAST can be used to calculate amino acid sequence identity, amino acid sequence homology, and nucleotide sequence identity. These calculations can be made along the entire length of each of the target sequences which are to be compared.

After two sequences have been aligned, a “percent sequence identity” can be determined. For these purposes, it is convenient to refer to a Reference Sequence and a Compared Sequence, where the Compared Sequence is compared to the Reference Sequence. Percent sequence identity can be determined according to the following formula: Percent Identity=100 [1−(C/R)], wherein C is the number of differences between the Reference Sequence and the Compared Sequence over the length of alignment between the Reference Sequence and the Compared Sequence where (i) each base or amino acid in the Reference Sequence that does not have a corresponding aligned base or amino acid in the Compared Sequence, (ii) each gap in the Reference Sequence, (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

Percent sequence identity can also be determined by other conventional methods, e.g., as described in Altschul et al., Bull. Math. Bio. 48: 603-616, 1986 and Henikoff and Henikoff, Proc. Natl. Acad. Sci. USA 89:10915-10919, 1992.

Specific Polynucleotide Probes

A polynucleotide of the present invention can comprise any continuous nucleotide sequence of Table 1, sequences which share sequence identity thereto, or complements thereof. The term “probe” refers to any substance that can be used to detect, identify, isolate, etc., another substance. A polynucleotide probe is comprised of nucleic acid can be used to detect, identify, etc., other nucleic acids, such as DNA and RNA.

These polynucleotides can be of any desired size that is effective to achieve the specificity desired. For example, a probe can be from about 7 or 8 nucleotides to several thousand nucleotides, depending upon its use and purpose. For instance, a probe used as a primer PCR can be shorter than a probe used in an ordered array of polynucleotide probes. Probe sizes vary, and the invention is not limited in any way by their size, e.g., probes can be from about 7-2000 nucleotides, 7-1000, 8-700, 8-600, 8-500, 8-400, 8-300, 8-150, 8-75, 7-50, 10-25, 14-16, at least about 8, at least about 10, at least about 15, at least about 25, etc. The polynucleotides can have non-naturally-occurring nucleotides, e.g., inosine, AZT, 3TC, etc. The polynucleotides can have 100% sequence identity or complementarity to a sequence of Table 1, or it can have mismatches or nucleotide substitutions, e.g., 1, 2, 3, 4, or 5 substitutions. The probes can be single-stranded or double-stranded.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for a differentially-expressed gene, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Another aspect of the present invention is a nucleotide sequence that is specific to, or for, a selective polynucleotide. The phrases “specific for” or “specific to” a polynucleotide have a functional meaning that the polynucleotide can be used to identify the presence of one or more target genes in a sample and distinguish them from non-target genes. It is specific in the sense that it can be used to detect polynucleotides above background noise (“non-specific binding”). A specific sequence is a defined order of nucleotides (or amino acid sequences, if it is a polypeptide sequence) which occurs in the polynucleotide, and which is characteristic of that target sequence, and substantially no non-target sequences. A probe or mixture of probes can comprise a sequence or sequences that are specific to a plurality of target sequences, e.g., where the sequence is a consensus sequence, a functional domain, etc., e.g., capable of recognizing a family of related genes. Such sequences can be used as probes in any of the methods described herein or incorporated by reference. Both sense and antisense nucleotide sequences are included. A specific polynucleotide according to the present invention can be determined routinely.

A polynucleotide comprising a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 90%, 95%, 99%, etc., identity (i.e., complementarity) to the probe, but less stringent conditions can also be used. A specific polynucleotide sequence can also be fused in-frame, at either its 5′ or 3′ end, to various nucleotide sequences as mentioned throughout the patent, including coding sequences for enzymes, detectable markers, GFP, etc, expression control sequences, etc.

A polynucleotide probe, especially one that is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of angiogenesis is desired, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Polynucleotide Composition

A polynucleotide according to the present invention can comprise, e.g., DNA, RNA, synthetic polynucleotide, peptide polynucleotide, modified nucleotides, dsDNA, ssDNA, ssRNA, dsRNA, and mixtures thereof. A polynucleotide can be single- or double-stranded, triplex, DNA:RNA, duplexes, comprise hairpins, and other secondary structures, etc. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. Nos. 5,411,863; 5,543,289; for instance, comprising ferromagnetic, super-magnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, and 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Nucleic Acid Detection Methods

Another aspect of the present invention relates to methods and processes for detecting a differentially-expressed gene. Detection methods have a variety of applications, including for diagnostic, prognostic, forensic, and research applications. To accomplish gene detection, a polynucleotide in accordance with the present invention can be used as a “probe.” The term “probe” or “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a gene or gene transcript present in a sample. Probes can be useful in a variety of ways, such as for diagnostic purposes, to identify homologs, and to detect, quantitate, or isolate a polynucleotide of the present invention in a test sample.

Assays can be utilized which permit quantification and/or presence/absence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the mentioned publications.

Many of such methods may require that the polynucleotide is labeled, or comprises a particular nucleotide type useful for detection. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

Detection can be desirable for a variety of different purposes, including research, diagnostic, prognostic, and forensic. For diagnostic purposes, it may be desirable to identify the presence or quantity of a polynucleotide sequence in a sample, where the sample is obtained from tissue, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide or polypeptide thereof can be used, including, e.g., blood, urine, saliva, stool (for extracting nucleic acid, see, e.g., U.S. Pat. No. 6,177,251), swabs comprising tissue, biopsied tissue, tissue sections, cultured cells, etc.

Detection can be accomplished in combination with polynucleotide probes for other genes, e.g., genes which are expressed in other disease states, tissues, cells, such as brain, heart, kidney, spleen, thymus, liver, stomach, small intestine, colon, muscle, lung, testis, placenta, pituitary, thyroid, skin, adrenal gland, pancreas, salivary gland, uterus, ovary, prostate gland, peripheral blood cells (T-cells, lymphocytes, etc.), embryo, breast, fat, adult and embryonic stem cells, specific cell-types, such as endothelial, epithelial, myocytes, adipose, etc.

Polynucleotides can be used in wide range of methods and compositions, including for detecting, diagnosing, staging, grading, assessing, prognosticating, etc. diseases and disorders associated with a differentially-expressed gene, for monitoring or assessing therapeutic and/or preventative measures, in ordered arrays, etc. Any method of detecting genes and polynucleotides of Table 1 can be used; certainly, the present invention is not to be limited how such methods are implemented.

Along these lines, the present invention relates to methods of detecting a differentially-expressed gene in a sample comprising nucleic acid. Such methods can comprise one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample, wherein said probe is a polynucleotide which is Table 1, a polynucleotide having, e.g., about 70%, 80%, 85%, 90%, 95%, 99%, or more sequence identity thereto, effective or specific fragments thereof, or complements thereto. The detection method can be applied to any sample, e.g., cultured primary, secondary, or established cell lines, tissue biopsy, blood, urine, stool, cerebral spinal fluid, and other bodily fluids, for any purpose.

Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

Generally, as used throughout the specification, the term “effective conditions” means, e.g., the particular milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complementarity. The effective conditions are selected such that the probe hybridizes to a pre-selected and/or definite target nucleic acid in the sample. For instance, if detection of a polynucleotide set forth in Table 1 is desired, a probe can be selected which can hybridize to such target gene under high stringent conditions, without significant hybridization to other genes in the sample. To detect homologs of a polynucleotide set forth in Table 1, the effective hybridization conditions can be less stringent, and/or the probe can comprise codon degeneracy, such that a homolog is detected in the sample.

As already mentioned, the methods can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of polynucleotides, the present invention also relates to determining the amounts at which polynucleotides of the present invention are expressed in sample and determining the differential expression of such polynucleotides in samples. Such methods can involve substantially the same steps as described above for presence/absence detection, e.g., contacting with probe, hybridizing, and detecting hybridized probe, but using more quantitative methods and/or comparisons to standards.

The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements. For further details, see the hybridization methods described above and below. Determining by such hybridization whether the target is differentially expressed (e.g., up-regulated or down-regulated) in the sample can also be accomplished by any effective means. For instance, the target's expression pattern in the sample can be compared to its pattern in a known standard, such as in a normal tissue, or it can be compared to another gene in the same sample. When a second sample is utilized for the comparison, it can be a sample of normal tissue that is known not to contain diseased cells. The comparison can be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal and sample, the sample is determined or diagnosed not to contain cells. However, if the ratio is different between the normal and sample tissues, the sample is determined to contain cancer cells. The approaches can be combined, and one or more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

Methods of Identifying Polymorphisms, Mutations, etc.

Polynucleotides of the present invention can also be utilized to identify mutant alleles, SNPs, gene rearrangements and modifications, and other polymorphisms of the wild-type gene. Mutant alleles, polymorphisms, SNPs, etc., can be identified and isolated from subjects with diseases that are known, or suspected to have, a genetic component. Identification of such genes can be carried out routinely (see, above for more guidance), e.g., using PCR, hybridization techniques, direct sequencing, mismatch reactions (see, e.g., above), RFLP analysis, SSCP (e.g., Orita et al., Proc. Natl. Acad. Sci., 86:2766, 1992), etc., where a polynucleotide, or fragment thereof, of Table 1 is used as a probe. The selected mutant alleles, SNPs, polymorphisms, etc., can be used diagnostically to determine whether a subject has, or is susceptible to a disorder associated with a gene of the present invention, as well as to design therapies and predict the outcome of the disorder. Methods involve, e.g., diagnosing a disorder associated with a gene of the present invention, or determining susceptibility to a disorder, comprising, detecting the presence of a mutation in a gene represented by a polynucleotide of the present invention, e.g., selected from Table 1 . The detecting can be carried out by any effective method, e.g., obtaining cells from a subject, determining the gene sequence or structure of a target gene (using, e.g., mRNA, cDNA, genomic DNA, etc), comparing the sequence or structure of the target gene to the structure of the normal gene, whereby a difference in sequence or structure indicates a mutation in the gene in the subject. Polynucleotides can also be used to test for mutations, SNPs, polymorphisms, etc., e.g., using mismatch DNA repair technology as described in U.S. Pat. Nos. 5,683,877; 5,656,430; Wu et al., Proc. Natl. Acad. Sci., 89:8779-8783, 1992.

The present invention also relates to methods of detecting polymorphisms in a gene of the present invention, comprising, e.g., comparing the structure of: genomic DNA comprising all or part of polymorphisms in a gene of the present invention, mRNA comprising all or part of polymorphisms in a gene of the present invention, CDNA comprising all or part of polymorphisms in a gene of the present invention, or a polypeptide comprising all or part of polymorphisms in a gene of the present invention, with the polynucleotide sequences represented by the GI or other accession numbers set forth in Table 1. The methods can be carried out on a sample from any source, e.g., cells, tissues, body fluids, blood, urine, stool, hair, egg, sperm,cerebral spinal fluid, etc.

These methods can be implemented in many different ways. For example, “comparing the structure” steps include, but are not limited to, comparing restriction maps, nucleotide sequences, amino acid sequences, RFLPs, Dnase sites, DNA methylation fingerprints (e.g., U.S. Pat. No. 6,214,556), protein cleavage sites, molecular weights, electrophoretic mobilities, charges, ion mobility, etc., between a standard [GENE] and a test [GENE]. The term “structure” can refer to any physical characteristics or configurations which can be used to distinguish between nucleic acids and polypeptides. The methods and instruments used to accomplish the comparing step depends upon the physical characteristics which are to be compared. Thus, various techniques are contemplated, including, e.g., sequencing machines (both amino acid and polynucleotide), electrophoresis, mass spectrometer (U.S. Pat. Nos. 6,093,541, 6,002,127), liquid chromatography, HPLC, etc.

To carry out such methods, “all or part” of the gene or polypeptide can be compared. For example, if nucleotide sequencing is utilized, the entire gene can be sequenced, including promoter, introns, and exons, or only parts of it can be sequenced and compared, e.g., exon 1, exon 2, etc.

Mutagenesis

Mutated polynucleotide sequences of the present invention are useful for various purposes, e.g., to create mutations of the polypeptides they encode, to identify functional regions of genomic DNA, to produce probes for screening libraries, etc. Mutagenesis can be carried out routinely according to any effective method, e.g., oligonucleotide-directed (Smith, M., Ann. Rev. Genet. 19:423-463, 1985), degenerate oligonucleotide-directed (Hill et al., Method Enzymology, 155:558-568, 1987), region-specific (Myers et al., Science, 229:242-246, 1985; Derbyshire et al., Gene, 46:145, 1986; Ner et al., DNA, 7:127, 1988), linker-scanning (McKnight and Kingsbury, Science, 217:316-324, 1982), directed using PCR, recursive ensemble mutagenesis (Arkin and Yourvan, Proc. Natl. Acad. Sci., 89:7811-7815, 1992), random mutagenesis (e.g., U.S. Pat. Nos. 5,096,815; 5,198,346; and 5,223,409), site-directed mutagenesis (e.g., Walder et al., Gene, 42:133, 1986; Bauer et al., Gene, 37:73, 1985; Craik, Bio Techniques, January 1985, 12-19; Smith et al., Genetic Engineering: Principles and Methods, Plenum Press, 1981), phage display (e.g., Lowman et al., Biochem. 30:10832-10837, 1991; Ladner et al., U.S. Pat. No. 5,223,409; Huse, WIPO Publication WO 92/06204), etc. Desired sequences can also be produced by the assembly of target sequences using mutually priming oligonucleotides (Uhlmann, Gene, 71:29-40, 1988). For directed mutagenesis methods, analysis of the three-dimensional structure of the a differentially-expressed gene polypeptide can be used to guide and facilitate making mutants which effect polypeptide activity. Sites of substrate-enzyme interaction or other biological activities can also be determined by analysis of crystal structure as determined by such techniques as nuclear magnetic resonance, crystallography or photoaffinity labeling. See, for example, de Vos et al., Science 255:306-312, 1992; Smith et al., J. Mol. Biol. 224:899-904, 1992; Wlodaver et al., FEBS Lett. 309:59-64, 1992.

In addition, libraries of differentially-expressed genes and fragments thereof can be used for screening and selection of gene variants. For instance, a library of coding sequences can be generated by treating a double-stranded DNA with a nuclease under conditions where the nicking occurs, e.g., only once per molecule, denaturing the double-stranded DNA, renaturing it to for double-stranded DNA that can include sense/antisense pairs from different nicked products, removing single-stranded portions from reformed duplexes by treatment with S1 nuclease, and ligating the resulting DNAs into an expression vector. By this method, xpression libraries can be made comprising “mutagenized” genes. The entire coding sequence or parts thereof can be used.

Polynucleotide Expression, Polypeptides Produced Thereby, and Specific-Binding Partners Thereto.

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve expression of a polypeptide coded for by the polynucleotide, to search for specific binding partners. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding polynucleotide is adjacent to a dhfr gene), cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, 293, endothelial, epithelial, muscle, embryonic and adult stem cells, ectodermal, mesenchymal, endodermal, neoplastic, blood, bovine CPAE (CCL-209), bovine FBHE (CRL-1395), human HUV-EC-C (CRL-1730), mouse SVEC4-10EHR1 (CRL-2161), mouse MS1 (CRL-2279), mouse MS1 VEGF (CRL-2460), insect cells, such as Sf9 (S. frugipeda) and Drosophila, bacteria, such as E. coli, Streptococcus, bacillus, yeast, such as Sacharomyces, S. cerevisiae, fungal cells, plant cells, embryonic or adult stem cells (e.g., mammalian, such as mouse or human).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Polynucleotide Res., 12(18):7035-7056, 1984; Dunn and Studier. J. Mol. Bio., 166:477-435, 1984; U.S. Pat. No. 5,891,636; Studier et al., Gene Expression Technology, Methods in Enzymology, 85:60-89, 1987. In addition, as discussed above, translational signals (including in-frame insertions) can be included.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

For expression and other purposes, a polynucleotide can contain codons found in a naturally-occurring gene, transcript, or CDNA, for example, e.g., as set forth in Table 1, or it can contain degenerate codons coding for the same amino acid sequences. For instance, it may be desirable to change the codons in the sequence to optimize the sequence for expression in a desired host. See, e.g., U.S. Pat. Nos. 5,567,600 and 5,567,862.

A polypeptide according to the present invention can be recovered from natural sources, transformed host cells (culture medium or cells) according to the usual methods, including, detergent extraction (e.g., non-ionic detergent, Triton X-100, CHAPS, octylglucoside, Igepal CA-630), ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis. Protein refolding steps can be used, as necessary, in completing the configuration of the mature protein. Finally, high performance liquid chromatography (HPLC) can be employed for purification steps. Another approach is express the polypeptide recombinantly with an affinity tag (Flag epitope, HA epitope, myc epitope, 6×His, maltose binding protein, chitinase, etc) and then purify by anti-tag antibody-conjugated affinity chromatography.

The present invention also relates to specific-binding partners. These include antibodies which are specific for polypeptides encoded by polynucleotides of the present invention, as well as other binding-partners which interact with polynucleotides and polypeptides of the present invention. Protein-protein interactions between polypeptides of the present invention and other polypeptides and binding partners can be identified using any suitable methods, e.g., protein binding assays (e.g., filtration assays, chromatography, etc.), yeast two-hybrid system (Fields and Song, Nature, 340: 245-247, 1989), protein arrays, gel-shift assays, FRET (fluorescence resonance energy transfer) assays, etc. Nucleic acid interactions (e.g., protein-DNA or protein-RNA) can be assessed using gel-shift assays, e.g., as carried out in U.S. Pat. No. 6,333,407 and 5,789,538.

Antibodies, e.g., polyclonal, monoclonal, recombinant, chimeric, humanized, single-chain, Fab, and fragments thereof, can be prepared according to any desired method. See, also, screening recombinant immunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. The antibodies can be IgM, IgG, subtypes, IgG2a, IgG1, etc. Antibodies, and immune responses, can also be generated by administering naked DNA See, e.g., U.S. Pat. Nos. 5,703,055; 5,589,466; 5,580,859. Antibodies can be used from any source, including, goat, rabbit, mouse, chicken (e.g., IgY; see, Duan, WO/029444 for methods of making antibodies in avian hosts, and harvesting the antibodies from the eggs). An antibody specific for a polypeptide means that the antibody recognizes a defined sequence of amino acids within or including the polypeptide. Other specific binding partners include, e.g., aptamers and PNA. antibodies can be prepared against specific epitopes or domains of a differentially-expressed gene.

The preparation of polyclonal antibodies is well-known to those skilled in the art. See, for example, Green et al., Production of Polyclonal Antisera, in IMMUNOCHEMICAL PROTOCOLS (Manson, ed.), pages 1-5 (Humana Press 1992); Coligan et al., Production of Polyclonal Antisera in Rabbits, Rats, Mice and Hamsters, in CURRENT PROTOCOLS IN IMMUNOLOGY, section 2.4.1 (1992). The preparation of monoclonal antibodies likewise is conventional. See, for example, Kohler & Milstein, Nature 256:495 (1975); Coligan et al., sections 2.5.1-2.6.7; and Harlow et al., ANTIBODIES: A LABORATORY MANUAL, page 726 (Cold Spring Harbor Pub. 1988).

Antibodies can also be humanized, e.g., where they are to be used therapeutically. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989), which is hereby incorporated in its entirety by reference. Techniques for producing humanized monoclonal antibodies are described, for example, in U.S. Pat. No. 6,054,297, Jones et al., Nature 321: 522 (1986); Riechmann et al., Nature 332: 323 (1988); Verhoeyen et al., Science 239: 1534 (1988); Carter et al., Proc. Nat'l Acad. Sci. USA 89: 4285 (1992); Sandhu, Crit. Rev. Biotech. 12: 437 (1992); and Singer et al., J. Immunol. 150: 2844 (1993).

Antibodies of the invention also may be derived from human antibody fragments isolated from a combinatorial immunoglobulin library. See, for example, Barbas et al., METHODS: A COMPANION TO METHODS IN ENZYMOLOGY, VOL. 2, page. 119 (1991); Winter et al., Ann. Rev. Immunol. 12: 433 (1994). Cloning and expression vectors that are useful for producing a human immunoglobulin phage library can be obtained commercially, for example, from STRATAGENE Cloning Systems (La Jolla, Calif.).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody-secreting hybridomas. Methods for obtaining human antibodies from transgenic mice are described, e.g., in Green et al., Nature Genet. 7:13 (1994); Lonberg et al., Nature 368:856 (1994); and Taylor et al., Int. Immunol. 6:579 (1994).

Antibody fragments of the present invention can be prepared by proteolytic hydrolysis of the antibody or by expression in E. coli of nucleic acid encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′).sub.2. This fragment can be further cleaved using a thiol reducing agent, and optionally a blocking group for the sulffiydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein. These patents are hereby incorporated in their entireties by reference. See also Nisoiihoff et al., Arch. Biochem. Biophys. 89:230 (1960); Porter, Biochem. J. 73:119 (1959); Edelman etal, METHODS IN ENZYMOLOGY, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques can also be used. For example, Fv fragments comprise an association of V.sub.H and V.sub.L chains. This association may be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, e.g., Sandhu, supra. Preferably, the Fv fragments comprise V.sub.H and V.sub.L chains connected by a peptide linker. These single-chain antigen binding proteins (sFv) are prepared by constructing a structural gene comprising nucleic acid sequences encoding the V.sub.H and V.sub.L domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by whitlow et al., METHODS: A COMPANION TO METHODS IN ENZYMOLOGY, VOL. 2, page 97 (1991); Bird etal.,Science 242:423-426 (1988); Ladneret al., U.S. Pat. No. 4,946,778; Pack et al., Bio/Technology 11: 1271-77 (1993); and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., METHODS: A COMPANION TO METHODS IN ENZYMOLOGY, VOL. 2, page 106 (1991).

The term “antibody” as used herein includes intact molecules as well as fragments thereof, such as Fab, F(ab′)2, and Fv which are capable of binding to an epitopic determinant present in Bin1 polypeptide. Such antibody fragments retain some ability to selectively bind with its antigen or receptor. The term “epitope” refers to an antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. Antibodies can be prepared against specific epitopes or polypeptide domains.

Antibodies which bind to differentially-expressed polypeptides of the present invention can be prepared using an intact polypeptide or fragments containing small peptides of interest as the immunizing antigen. For example, it may be desirable to produce antibodies that specifically bind to the N- or C-terminal domains of differentially-expressed polypeptide. The polypeptide or peptide used to immunize an animal which is derived from translated cDNA or chemically synthesized which can be conjugated to a carrier protein, if desired. Such commonly used carriers which are chemically coupled to the immunizing peptide include keyhole limpet hemocyanin (KLH), thyroglobulin, bovine serum albumin (BSA), and tetanus toxoid.

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Imnmunology, Wiley Interscience, 1994, incorporated by reference).

Anti-idiotype technology can also be used to produce invention monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

Methods of Detecting Polypeptides

Polypeptides coded for by genes of the present invention can be detected, visualized, determined, quantitated, etc. according to any effective method. useful methods include, e.g., but are not limited to, immunoassays, RIA (radioimmunassay), ELISA, (enzyme-linked-immunosorbent assay), immunoflourescence, flow cytometry, histology, electron microscopy, light microscopy, in situ assays, immunoprecipitation, Western blot, etc Immunoassays may be carried in liquid or on biological support. For instance, a sample (e.g., blood, stool, urine, cells, tissue, body fluids, etc.) can be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled gene specific antibody. The solid phase support can then be washed with a buffer a second time to remove unbound antibody. The amount of bound label on solid support may then be detected by conventional means.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, polyacrylamides, and magnetite. A support material can have any structural or physical configuration. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads

One of the many ways in which gene peptide-specific antibody can be detectably labeled is by linking it to an enzyme and using it in an enzyme immunoassay (EIA). See, e.g., Voller, A., “The Enzyme Linked Immunosorbent Assay (ELISA),” 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, .alpha.-glycerophosphate, dehydrogenase, triose phosphate isomerase, horseradish peroxidase, alkaline phosphatase, asparaginase, glucose oxidase, beta.-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect peptides through the use of a radioimmunoassay (RIA). See, e.g., Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as those in the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Diagnostic

The present invention also relates to methods and compositions for diagnosing a disorder associated with angiogenesis, or determining susceptibility to a vascular disorder, using polynucleotides, polypeptides, and specific-binding partners of the present invention to detect, assess, determine, etc., differentially-expressed genes and the polypeptides they encode. In such methods, the gene can serve as a marker for the disorder, e.g., where the gene, when mutant, is a direct cause of the disorder; where the gene is affected by another gene(s) which is directly responsible for the disorder, e.g., when the gene is part of the same signaling pathway as the directly responsible gene; where the gene is chromosomally linked to the gene(s) directly responsible for the disorder, and segregates with it, when the gene is differentially-expressed when the disorder is present (e.g., angiogenesis is associated with cancer; a cancer sample that contains new blood vessels will show expressed of genes, such as those showing a U1T or U1S profile). Many other situations are possible. To detect, assess, determine, etc., a probe specific for the gene can be employed as described above and below. Any method of detecting and/or assessing the gene can be used, including detecting expression of the gene using polynucleotides, antibodies, or other specific-binding partners.

The present invention relates to methods of diagnosing a disorder associated with expression of a gene selected from Table 1, or determining a subject's susceptibility to such disorder, comprising, e.g., assessing the expression of the gene in a tissue sample comprising tissue or cells suspected of having the disorder (e.g., where the sample comprises cells capable of forming blood vessels. The phrase “diagnosing” indicates that it is determined whether the sample has the disorder. A “disorder” means, e.g., any abnormal condition as in a disease or malady. “Determining a subject's susceptibility to a disease or disorder” indicates that the subject is assessed for whether s/he is predisposed to get such a disease or disorder, where the predisposition is indicated by abnormal expression of the gene (e.g., gene mutation, gene expression pattern is not normal, etc.). Predisposition or susceptibility to a disease may result when a such disease is influenced by epigenetic, environmental, etc., factors. Such diseases include, e.g., inflammatory diseases, such as rheumatoid arthritis, osteoarthritis, asthma, pulmonary fibrosis, age-related macular degeneration (ARMD), diabetic retinopathy, macular degeneration, and retinopathy of prematurity (ROP), endometriosis, cancer, Coats' disease, peripheral retinal neovascularization, neovascular glaucoma, psoriasis, retrolental fibroplasias, angiofibroma, inflammation, etc. Diagnosing includes prenatal screening where samples from the fetus or embryo (e.g., via amniocentesis or CV sampling) are analyzed for the expression of the gene.

By the phrase “assessing expression of a gene,” it is meant that the functional status of the gene is evaluated. This includes, but is not limited to, measuring expression levels of said gene, determining the genomic structure of said gene, determining the mRNA structure of transcripts from said gene, or measuring the expression levels of polypeptide coded for by said gene. Thus, the term “assessing expression” includes evaluating the all aspects of the transcriptional and translational machinery of the gene. For instance, if a promoter defect causes, or is suspected of causing, the disorder, then a sample can be evaluated (i.e., “assessed”) by looking (e.g., sequencing or restriction mapping) at the promoter sequence in the gene, by detecting transcription products (e.g., RNA), by detecting translation product (e.g., polypeptide). Any measure of whether the gene is functional can be used, including, polypeptide, polynucleotide, and functional assays for the gene's biological activity.

In making the assessment, it can be useful to compare the results to a gene which is not associated with the disorder, e.g., a gene which is not differentially-expressed during angiogenesis. The nature of the comparison can be determined routinely, depending upon how the assessing is accomplished. If, for example, the mRNA levels of a sample is detected, then the mRNA levels of a normal can serve as a comparison, or a gene which is known not to be affected by the disorder. Methods of detecting mRNA are well known, and discussed above, e.g., but not limited to, Northern blot analysis, polymerase chain reaction (PCR), reverse transcriptase PCR, RACE PCR, etc. Similarly, if polypeptide production is used to evaluate the gene, then the polypeptide in a normal tissue sample can be used as a comparison, or, polypeptide from a different gene whose expression is known not to be affected by the disorder. These are only examples of how such a method could be carried out.

The genes and polypeptides of the present invention can be used to identify, detect, stage, determine the presence of, prognosticate, treat, study, etc., diseases and conditions of associated with angiogenesis as mentioned above. The present invention relates to methods of identifying a genetic basis for a disease or disease-susceptibility, comprising, e.g., determining the association of a vascular disease or disease-susceptibility with a gene of the present invention. An association between a disease or disease-susceptibility and nucleotide sequence includes, e.g., establishing (or finding) a correlation (or relationship) between a DNA marker (e.g., gene, VNTR, polymorphism, EST, etc.) and a particular disease state. Once a relationship is identified, the DNA marker can be utilized in diagnostic tests and as a drug target. Any region of the gene can be used as a source of the DNA marker, exons, introns, intergenic regions, etc.

Human linkage maps can be constructed to establish a relationship between a gene and a vascular disease or condition. Typically, polymorphic molecular markers (e.g., STRP's, SNP's, RFLP's, VNTR's) are identified within the region, linkage and map distance between the markers is then established, and then linkage is established between phenotype and the various individual molecular markers. Maps can be produced for an individual family, selected populations, patient populations, etc. In general, these methods involve identifying a marker associated with the disease (e.g., identifying a polymorphism in a family which is linked to the disease) and then analyzing the surrounding DNA to identity the gene responsible for the phenotype. See, e.g., Kruglyak et al., Am. J. Hum. Genet., 58, 1347-1363, 1996; Matise et al., Nat. Genet., 6(4):384-90, 1994.

Assessing the effects of therapeutic and preventative interventions (e.g., administration of a drug, chemotherapy, radiation, etc.) on vascular or angiogenic disorders is a major effort in drug discovery, clinical medicine, and pharmacogenomics. The evaluation of therapeutic and preventative measures, whether experimental or already in clinical use, has broad applicability, e.g., in clinical trials, for monitoring the status of a patient, for analyzing and assessing animal models, and in any scenario involving cancer treatment and prevention. Analyzing the expression profiles of polynucleotides of the present invention can be utilized as a parameter by which interventions are judged and measured. Treatment of a disorder can change the expression profile in some manner which is prognostic or indicative of the drug's effect on it. Changes in the profile can indicate, e.g., drug toxicity, return to a normal level, etc. Accordingly, the present invention also relates to methods of monitoring or assessing a therapeutic or preventative measure (e.g., chemotherapy, radiation, anti-neoplastic drugs, antibodies, etc.) in a subject having a vascular or angiogenic disorder, or, susceptible to such a disorder, comprising, e.g., detecting the expression levels of differentially-expressed gene or a polypeptide it encodes. A subject can be a cell-based assay system, non-human animal model, human patient, etc. Detecting can be accomplished as described for the methods above and below. By “therapeutic or preventative intervention,” it is meant, e.g., a drug administered to a patient, surgery, radiation, chemotherapy, and other measures taken to prevent, treat, or diagnose a disorder.

Expression can be assessed in any sample comprising any tissue or cell type, body fluid, etc., as discussed for other methods of the present invention, including cells which are capable of forming blood vessels, e.g., stem cells, endothelial cells, etc.

The present invention also relates to methods of using binding partners, such as antibodies, to deliver active agents to vascular tissue for a variety of different purposes, including, e.g., for diagnostic, therapeutic (e.g., to treat diseases associated with excessive angiogenesis or to treat cancer), and research purposes. Methods can involve delivering or administering an active agent to the vascular tissue, comprising, e.g., administering to a subject in need thereof, an effective amount of an active agent coupled to a binding partner specific for a human polypeptide of Table 1, especially cell-surface polypeptides, wherein said binding partner is effective to deliver said active agent to said vascular tissue.

Any type of active agent can be used, including, therapeutic, cytotoxic, cytostatic, chemotherapeutic, anti-neoplastic, anti-proliferative, anti-biotic, etc., agents. A chemotherapeutic agent can be, e.g., DNA-interactive agent, alkylating agent, antimetabolite, tubulin-interactive agent, hormonal agent, hydroxyurea, Cisplatin, Cyclophosphamide, Altretamine, Bleomycin, Dactinomycin, Doxorubicin, Etoposide, Teniposide, paclitaxel, cytoxan, 2-methoxycarbonylaminobenzimidazole, Plicamycin, Methotrexate, Fluorouracil, Fluorodeoxyuridin, CB3717, Azacitidine, Floxuridine, Mercapyopurine, 6-Thioguanine, Pentostatin, Cytarabine, Fludarabine, etc. Agents can also be contrast agents useful in imaging technology, e.g., X-ray, CT, CAT, MRI, ultrasound, PET, SPECT, and scintographic.

An active agent can be associated in any manner with a binding partner which is effective to achieve its delivery specifically to the target. Specific delivery or targeting indicates that the agent is provided to the target tissue, without being substantially provided to other tissues. This is useful especially where an agent is toxic, and specific targeting to sites of angiogenesis, enables the majority of the toxicity to be aimed at such sites, with as small as possible effect on other tissues in the body. The association of the active agent and the binding partner (“coupling) can be direct, e.g., through chemical bonds between the binding partner and the agent, or, via a linking agent, or the association can be less direct, e.g., where the active agent is in a liposome, or other carrier, and the binding partner is associated with the liposome surface. In such case, the binding partner can be oriented in such a way that it is able to bind to polypeptide on the cell surface. Methods for delivery of DNA via a cell-surface receptor is described, e.g., in U.S. Pat. No. 6,339,139.

Methods of Detecting Angiogenesis

The present invention also relates to detecting the presence and/or extent of blood vessels in a sample. The detected blood vessels can be established or pre-existing vessels, newly formed vessels, vessels in the process of forming, or combinations thereof. A blood vessel includes any biological structure that conducts blood, including arteries, veins, capillaries, microvessels, vessel lumen, endothelial-lined sinuses, etc. These methods are useful for a variety of purposes. In cancer, for instance, the extent of vascularization can be an important factor in determining the clinical behavior of neoplastic cells. See, e.g., Weidner et al., N. Engl. J. Med., 324:1-8, 1991. Thus, the presence and extent of blood vessels, including the angiogenic process itself, can be useful for the diagnosis, prognosis, treatment, etc., of cancer and other neoplasms. Detection of vessels can also be utilized for the diagnosis, prognosis, treatment, of any diseases or conditions associated with vessel growth and production, to assess agents which modulate angiogenesis, to assess angiogenic gene therapy, etc. The term “vascular tissue” can also be used to describe blood vessels.

An example of a method of detecting the presence or extent of blood vessels in a sample is determining an angiogenic index of a tissue or cell sample comprising, e.g., assessing in a sample, the expression levels of differentially-expressed or -regulated genes selected from Table 1, whereby said levels are indicative of the angiogenic index. By the phrase “angiogenic index,” it is meant the extent or degree of vascularity of the tissue, e.g., the number or amount of blood vessels in the sample of interest. Amounts of nucleic acid or polypeptide can be assessed (e.g., determined, detected, etc.) by any suitable method. There is no limitation on how detection is performed.

For instance, if nucleic acid is to be assessed, e.g., an mRNA corresponding to a differentially-expressed gene, the methods for detecting it, assessing its presence and/or amount, can be determined by any the methods mentioned above, e.g., nucleic acid based detection methods, such as Northern blot analysis, RT-PCR, RACE, differential display, NASBA and other transcription based amplification systems, polynucleotide arrays, etc. If RT-PCR is employed, cDNA can be prepared from the mRNA extracted from a sample of interest. Once the cDNA is obtained, PCR can be employed using oligonucleotide primer pairs that are specific for a differentially-expressed gene. The specific probes can be of a single sequence, or they can be a combination of different sequences. A polynucleotide array can also be used to assess nucleic, e.g., where the RNA of the sample of interest is labeled (e.g., using a transcription based amplification method, such as U.S. Pat. No. 5,716,785) and then hybridized to probe fixed to a solid substrate.

Polypeptide detection can also be carried out by any available method, e.g., by Western blots, ELISA, dot blot, immunoprecipitation, RIA, immunohistochemistry, etc. For instance, a tissue section can be prepared and labeled with a specific antibody (indirect or direct), visualized with a microscope, and then the number of vessels in a particular field of view counted, where staining with antibody is used to identify and count the vessels. Amount of a polypeptide can be quantitated without visualization, e.g., by preparing a lysate of a sample of interest, and then determining by ELISA or Western the amount of polypeptide per quantity of tissue. Again, there is no limitation on how detection is performed.

In addition to assessing the angiogenic index using an antibody specific for a differentially-expressed gene, other methods of determining tissue vascularity can be applied. Tissue vascularity is typically determined by assessing the number and density of vesssels present in a given sample. For example, microvessel density (MVD) can be estimated by counting the number of endothelial clusters in a high-power microscopic field, or detecting a marker specific for microvascular endothelium or other markers of growing or established blood vessels, such as CD31 (also known as platelet-endothelial cell adhesion molecule or PECAM). A CD31 antibody can be employed in conventional immunohistological methods to immunostain tissue sections as described by, e.g., Penfold et al., Br. J. Oral and Maxill. Surg., 34: 37-41; U.S. Pat. No. 6,017,949; Dellas et al., Gyn. Oncol., 67:27-33, 1997; and others.

The methods can be performed with as many differentially-regulated or expressed genes as necessary or desired, e.g., at least two, at least five, at least 10, at least 20, etc. The methods can also be performed with one or more classes of genes, e.g., sustained up-regulated genes, sustained down-regulated genes, nuclear regulatory factors, cell surface markers, ECM, protein manufacture, protein degradation, cell signaling, and/or endothelial cell markers, any of the expression patterns described herein, and combinations thereof. Because a differentially-expressed gene may not be angiogenic-specific, the pattern of a set of genes can be useful to assess the status of the tissue, especially in terms of whether neoangiogenesis is occurring. For instance, to assess angiogenesis in a sample, a set of sustained differentially expressed genes can be selected from different functional categories, such as nuclear regulatory factors, muscle markers (e.g., for fully functional and advanced vessels), protein degradation markers, etc., for genes which are expressed at the 24-hour period when functional tubes are formed.

In addition to a differentially-expressed gene, other genes and their corresponding products can be detected. For instance, it may be desired to detect a gene which is expressed ubiquitously in the sample. A ubiquitously expressed gene, or product thereof, is present in all cell types, e.g., in about the same amount, e.g., beta-actin. Similarly, a gene or polypeptide that is expressed selectively in the tissue or cell of interest can be detected. A selective gene or polypeptide is characteristic of the tissue or cell-type in which it is made. This can mean that it is expressed only in the tissue or cell, and in no other tissue- or cell-type, or it can mean that it is expressed preferentially, differentially, and more abundantly (e.g., at least 5-fold, 10-fold, etc., or more) when compared to other types. The expression of the ubiquitous or selective gene or gene product can be used as a control or reference marker to compare to the expression of differentially-expression genes. Typically, expression of the gene can be assessed by detecting mRNA produced from it. Other markers for blood vessels and angiogenesis can also be detected, such as angiogenesis-related genes or polypeptides. By the phrase “angiogenesis-related,” it is meant that it is associated with blood vessels and therefore indicative of their presence. There are a number of different genes and gene products that are angiogenesis-related, e.g., Vezf1 (e.g., Xiang et al., Dev. Bio., 206:123-141, 1999), VEGF, VEGF receptors (such as KDR/Flk-1), angiopoietin, Tie-1 and Tie-2 (e.g., Sato et al., Nature, 376:70-74, 1995), PECAM-1 or CD31 (e.g., DAKO, Glostrup. Denmark), CD34, factor VIII-related antigen (e.g., Brustmann et al., Gyn. Oncol., 67:20-26, 1997).

Identifying Agent Methods and Modulating Angiogenesis

The present invention also relates to methods of identifying agents, and the agents themselves, which modulate differentially-expressed genes or polypeptides expressed in endothelial or other angiogenic-forming cells. These agents can be used to modulate the biological activity of the polypeptide encoded for the gene, or the gene, itself. Agents which regulate the gene or its product are useful in variety of different environments, including as medicinal agents to treat or prevent disorders associated with angiogenesis and as research reagents to modify the function of tissues and cell.

Methods of identifying agents generally comprise steps in which an agent is placed in contact with the gene, its transcription product, its translation product, or other target, and then a determination is performed to assess whether the agent “modulates” the target. The specific method utilized will depend upon a number of factors, including, e.g., the target (i.e., is it the gene or polypeptide encoded by it), the environment (e.g., in vitro or in vivo), the composition of the agent, etc.

For modulating the expression of a gene, a method can comprise, in any effective order, one or more of the following steps, e.g., contacting a gene (e.g., in a cell population) with a test agent under conditions effective for said test agent to modulate the expression of it, and determining whether said test agent modulates said gene. An agent can modulate expression of a gene at any level, including transcription (e.g., by modulating the promoter), translation, and/or perdurance of the nucleic acid (e.g., degradation, stability, etc.) in the cell.

For modulating the biological activity of polypeptides, a method can comprise, in any effective order, one or more of the following steps, e.g., contacting a polypeptide (e.g., in a cell, lysate, or isolated) with a test agent under conditions effective for said test agent to modulate the biological activity of said polypeptide, and determining whether said test agent modulates said biological activity.

Contacting the gene or polypeptide with the test agent can be accomplished by any suitable method and/or means that places the agent in a position to functionally control expression or biological activity of the gene or its product in the sample. Functional control indicates that the agent can exert its physiological effect through whatever mechanism it works. The choice of the method and/or means can depend upon the nature of the agent and the condition and type of environment in which the gene or its product is presented, e.g., lysate, isolated, or in a cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

Agents can be directed to, or targeted to, any part of the polypeptide which is effective for modulating it. For example, agents, such as antibodies and small molecules, can be targeted to cell-surface, exposed, extracellular, ligand binding, functional, etc., domains of the polypeptide. Agents can also be directed to intracellular regions and domains, e.g., regions where the polypeptide couples or interacts with intracellular or intramembrane binding partners.

After the agent has been administered in such a way that it can gain access to the gene or gene product (including DNA, mRNA, and polypeptides), it can be determined whether the test agent modulates its expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate gene expression means, e.g., that the test agent has-an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the agent. This effect includes, increase, decrease, block, inhibit, enhance, etc.

A test agent can be of any molecular composition, e.g., chemical compounds, biomolecules, such as polypeptides, lipids, nucleic acids (e.g., antisense to a polynucleotide) carbohydrates, antibodies, ribozymes, double-stranded RNA, aptamers, etc. For example, if a polypeptide to be modulated is a cell-surface molecule, a test agent can be an antibody that specifically recognizes it and, e.g., causes the polypeptide to be internalized, leading to its down regulation on the surface of the cell. Such an effect does not have to be permanent, but can require the presence of the antibody to continue the down-regulatory effect. Antibodies can also be used to modulate the biological activity of a polypeptide in a lysate or other cell-free form.

The present invention also relates to methods of identifying modulators of a gene, differentially-expressed during angiogenesis, in a cell population capable of forming blood vessels, comprising, one or more of the following steps in any effective order, e.g., contacting the cell population with a test agent under conditions effective for said test agent to modulate the to modulate a differentially-expressed gene selected from Table 1, or a polypeptide thereof. These methods are useful, e.g., for drug discovery in identifying and confirming the angiogenic activity of agents, for identifying molecules in the normal pathway of angiogenesis, etc.

Any cell population capable of forming blood vessels can be utilized. Useful models, included those mentioned above, e.g., in vivo Matrigel-type assays, tumor neovascularization assays, CAM assays, BCE assays, migration assays, HUVEC growth inhibition assays, animal models (e.g., tumor growth in athymic mice), models involving hybrid cell and electronic-based components, etc. Cells can include, e.g., endothelial, epithelial, muscle, embryonic and adult stem cells, ectodermal, mesenchymal, endodermal, neoplastic, blood, bovine CPAE (CCL-209), bovine FBHE (CRL-1395), human HUV-EC-C (CRL-1730), mouse SVEC4-10EHR1 (CRL-2161), mouse MS1 (CRL-2279), mouse MS1 VEGF (CRL-2460), stem cells, etc. The phrase “capable of forming blood vessels” does not indicate a particular cell-type, but simply that the cells in the population are able under appropriate conditions to form blood vessels. In some circumstances, the population may be heterogeneous, comprising more than one cell-type, only some which actually differentiate into blood vessels, but others which are necessary to initiate, maintain, etc., the process of vessel formation.

The cell population can be contacted with the test agent in any manner and under any conditions suitable for it to exert an effect on the cells, and to modulate the differentially-expressed gene or polypeptide. The means by which the test agent is delivered to the cells may depend upon the type of test agent, e.g., its chemical nature, and the nature of the cell population. Generally, a test agent must have access to the cell population, so it must be delivered in a form (or pro-form) that the population can experience physiologically, i.e., to put in contact with the cells. For instance, if the intent is for the agent to enter the cell, if necessary, it can be associated with any means that facilitate or enhance cell penetrance, e.g., associated with antibodies or other reagents specific for cell-surface antigens, liposomes, lipids, chelating agents, targeting moieties, etc. Cells can also be treated, manipulated, etc., to enhance delivery, e.g., by electroporation, pressure variation, etc.

A purpose of administering or delivering the test agents to cells capable of forming blood vessels is to determine whether they modulate a gene of Table 1 or a polypeptide thereof. By the phrase “modulate,” it is meant that the gene or polypeptide affects the polypeptide or gene in some way. Modulation includes effects on transcription, RNA splicing, RNA editing, transcript stability and turnover, translation, polypeptide activity, and, in general, any process involved in the expression and production of the gene and gene product. The modulatory activity can be in any direction, and in any amount, including, up, down, enhance, increase, stimulate, activate, induce, turn on, turn off, decrease, block, inhibit, suppress, prevent, etc.

Any type of test agent can be used, comprising any material, such as chemical compounds, biomolecules, such as polypeptides (including polypeptide fragments and mimics), lipids, nucleic acids, carbohydrates, antibodies, small molecules, fusion proteins, etc. Test agents include, e.g., protamine (Taylor et al., Nature, 297:307, 1982), heparins, steroids, such as tetrahydrocortisol, which lack gluco- and mineral-corticoid activity (e.g., Folkman et al., Science 221:719, 1983 and U.S. Pat. Nos. 5,001,116 and 4,994,443), angiostatins (e.g., WO 95/292420), triazines (e.g., U.S. Pat. No. 6,150,362), thrombospondins, endostatins, platelet factor 4, fumagillin-derivate AGH 1470, alpha-interferon, quinazolinones (e.g., U.S. Pat. No. 6,090,814), substituted dibenzothiophenes (e.g., U.S. Pat. No. 6,022,307), deoxytetracyclines, cytokines, chemokines, FGFs, etc.

Whether the test agent modulates a gene or polypeptide can be determined by any suitable method. These methods include, detecting gene transcription, detecting mRNA, detecting polypeptide and activity thereof. The detection methods includes those mentioned herein, e.g., PCR, RT-PCR, Northern blot, ELISA, Western, RIA, etc. In addition to detecting nucleic acid and polypeptide, further downstream targets can be used to assess the effects of modulators, including, the presence or absence of neoangiogenesis (e.g., using any of the mentioned test systems, such as CAM, BCE, in vivo Matrigel-type assays) as modulated by a test agent.

The present invention also relates to methods of regulating angiogenesis in a system comprising cells, comprising administering to the system an effective amount of a modulator of a differentially-expressed gene or polypeptide under conditions effective for the modulator to modulate the gene or polypeptide, whereby angiogenesis is regulated. A system comprising cells can be an in vivo system, such as a heart or limb present in a patient (e.g., angiogenic therapy to treat myocardial infarction), isolated organs, tissues, or cells, in vitro assays systems (CAM, BCE, etc), animal models (e.g., in vivo, subcutaneous, chronically ischemic lower limb in a rabbit model, cancer models), hosts in need of treatment (e.g., hosts suffering from angiogenesis related diseases, such as cancer, ischemic syndromes, arterial obstructive disease, to promote collateral circulation, to promote vessel growth into bioengineered tissues, etc.

A modulator useful in such method are those mentioned already, e.g., nucleic acid (such as an anti-sense to a gene to disrupt transcription or translation of the gene), antibodies (e.g., to inhibit a cell-surface protein, such as an antibody specific-for the extracellular domain). Antibodies and other agents which target a polypeptide can be conjugated to a cytotoxic or cytostatic agent, such as those mentioned already. A modulator can also be a differentially-expressed gene, itself, e.g., when it is desired to deliver the polypeptide to cells analogously to gene therapy methods. A complete gene, or a coding sequence operably linked to an expression control sequence (i.e., an expressible gene) can be used to produce polypeptide in the target cells.

By the phrase “regulating angiogenesis,” it is meant that angiogenesis is effected in a desired way by the modulator. This includes, inhibiting, blocking, reducing, stimulating, inducing, etc., the formation of blood vessels. For instance, in cancer, where the growth of new blood vessels is undesirable, modulators of a differentially-expressed can be used to inhibit their formation, thereby treating the cancer. Such inhibitory modulators include, e.g., antibodies to the extracellular regions of a differentially-expressed polypeptide, and, antisense RNA to inhibit translation of a differentially-expressed mRNA into polypeptide (for guidance on administering and designing anti-sense, see, e.g., U.S. Pat. Nos. 6,153,595, 6,133,246, 6,117,847, 6,096,722, 6,087,343, 6,040,296, 6,005,095, 5,998,383, 5,994,230, 5,891,725, 5,885,970, and 5,840,708). On the other hand, angiogenesis can be stimulated to treat ischemic syndromes and arterial obstructive disease, to promote collateral circulation, and to promote vessel growth into bioengineered tissues, etc., by administering the a differentially-expressed gene or polypeptide to a target cell population.

Markers

The polynucleotides of the present invention can be used with other markers, especially angiogenic markers, to identity, detect, stage, diagnosis, determine, prognosticate, treat, etc., tissue, diseases and conditions, etc, associated with angiogenesis. Markers can be polynucleotides, polypeptides, antibodies, ligands, specific binding partners, etc. The targets for such markers include, but are not limited genes and polypeptides that are selective for angiogenesis.

Therapeutics

Selective polynucleotides, polypeptides, and specific-binding partners thereto, can be utilized in therapeutic applications, especially to treat diseases and conditions associated with abnormal, insufficient, excessive, angiogenesis. Useful methods include, but are not limited to, immunotherapy (e.g., using specific-binding partners to polypeptides), vaccination (e.g., using a selective polypeptide or a naked DNA encoding such polypeptide), protein or polypeptide replacement therapy, gene therapy (e.g., germ-line correction, antisense), etc.

Various immunotherapeutic approaches can be used. For instance, unlabeled antibody that specifically recognizes an antigen can be used to stimulate the body to destroy or attack vascular tissue. e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

An antibody or other specific-binding partner can be conjugated to a second molecule, such as a cytotoxic agent, and used for targeting the second molecule to a tissue-antigen positive cell (Vitetta, E. S. et al., 1993, Immunotoxin therapy, in DeVita, Jr., V. T. et al., eds, Cancer: Principles and Practice of Oncology, 4th ed., J. B. Lippincott Co., Philadelphia, 2624-2636). Examples of cytotoxic agents include, but are not limited to, antimetabolites, alkylating agents, anthracyclines, antibiotics, anti-mitotic agents, radioisotopes and chemotherapeutic agents. Further examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, 1-dehydrotestosterone, diptheria toxin, Pseudomonas exotoxin (PE) A, PE40, abrin, elongation factor-2 and glucocorticoid. Techniques for conjugating therapeutic agents to antibodies are well.

In addition to immunotherapy, polynucleotides and polypeptides can be used as targets for non-immunotherapeutic applications, e.g., using compounds which interfere with function, expression (e.g., antisense as a therapeutic agent), assembly, etc. RNA interference can be used in vitro and in vivo to silence a gene when its expression contributes to angiogenesis (but also for other purposes, e.g., to identify the gene's function to change a developmental pathway of a cell, etc.). See, e.g., Sharp and Zamore, Science, 287:2431-2433, 2001; Grishok et al., Science, 287:2494, 2001.

Delivery of therapeutic agents can be achieved according to any effective method, including, liposomes, viruses, plasmid vectors, bacterial delivery systems, orally, systemically, etc. Therapeutic agents of the present invention can be administered in any form by any effective route, including, e.g., oral, parenteral, enteral, intraperitoneal, topical, transdermal (e.g., using any standard patch), intravenously, ophthalmic, nasally, local, non-oral, such as aerosol, inhalation, subcutaneous, intramuscular, buccal, sublingual, rectal, vaginal, intra-arterial, and intrathecal, etc. They can be administered alone, or in combination with any ingredient(s), active or inactive.

In addition to therapeutics, per se, the present invention also relates to methods of treating a vascular disease or a disease association with vascularization, comprising, e.g., administering to a subject in need thereof a therapeutic agent which is effective for regulating expression of a gene, or polypeptide encoded thereby, selected from the differentially-expressed genes set forth in Table 1, wherein said therapeutic agent modulates angiogenesis. The term “treating” is used conventionally, e.g., the management or care of a subject for the purpose of combating, alleviating, reducing, relieving, improving the condition of, etc., of a disease or disorder. Diseases or disorders which can be treated in accordance with the present invention include, but are not limited to inflammatory diseases, such as rheumatoid arthritis, osteoarthritis, asthma, pulmonary fibrosis, age-related macular degeneration (ARMD), diabetic retinopathy, macular degeneration, and retinopathy of prematurity (ROP), endometriosis, cancer, Coats' disease, peripheral retinal neovascularization, neovascular glaucoma, psoriasis, retrolental fibroplasias, angiofibroma, inflammation, etc.

Any agent which “treats” the disease can be used. Such an agent can be one which regulates the expression of a differentially-expressed gene or polypeptide. Expression refers to the same acts already mentioned, e.g. transcription, translation, splicing, stability of the mRNA or protein product, activity of the gene product, differential expression, etc. For instance, if the condition was a result of a complete deficiency of the gene product, administration of gene product to a patient would be said to treat the disease and regulate the gene's expression. Many other possible situations are possible, e.g., where the gene is aberrantly expressed, and the therapeutic agent regulates the aberrant expression by restoring its normal expression pattern.

Antisense

Antisense polynucleotide (e.g., RNA) can also be prepared from a polynucleotide according to the present invention, preferably an anti-sense to a sequence of Table 1 . Antisense polynucleotide can be used in various ways, such as to regulate or modulate expression of the polypeptides they encode, e.g., inhibit their expression, for in situ hybridization, for therapeutic purposes, for making targeted mutations (in vivo, triplex, etc.) etc. For guidance on administering and designing anti-sense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708. An antisense polynucleotides can be operably linked to an expression control sequence. A total length of about 35 bp can be used in cell culture with cationic liposomes to facilitate cellular uptake, but for in vivo use, preferably shorter oligonucleotides are administered, e.g. 25 nucleotides.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); 4,973,679; Sproat et al., “2′-O-Methyloligoribonucleotides: synthesis and applications,” Oligonucleotides and Analogs: A Practical Approach, Eckstein (ed.), IRL Press, Oxford, 1991, 49-86; Iribarren et al., “2′O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 1991, 19, 2629-2635.

Arrays

The present invention also relates to an ordered array of polynucleotide probes and specific-binding partners (e.g., antibodies) for detecting the expression of a differentially-regulated genes in a sample, comprising, one or more polynucleotide probes or specific binding partners associated with a solid support, wherein each probe is specific for said gene, and the probes comprise a nucleotide sequence of Table 1 which is specific for said gene, a nucleotide sequence having sequence identity to Table 1 which is specific for said gene or polynucleotide, or complements thereto, or a specific-binding partner which is specific for said differentially-regulated gene.

The phrase “ordered array” indicates that the probes are arranged in an identifiable or position-addressable pattern, e.g., such as the arrays disclosed in U.S. Pat. Nos. 6,156,501, 6,077,673, 6,054,270, 5,723,320, 5,700,637, WO09919711, WO00023803. The probes are associated with the solid support in any effective way. For instance, the probes can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes can fill the hollow orifice, be absorbed into the solid filament, be attached to the surface of the orifice, etc. Probes can be of any effective size, sequence identity, composition, etc., as already discussed.

Ordered arrays can further comprise polynucleotide probes or specific-binding partners which are specific for other genes, including genes specific for a particular tissue-type or phenotype. Arrays can be provided which contain specific sets of genes, e.g., U1S, U8S, U1T, U8T, D1S, D8S, D1T, D8T, functional groups, such as nuclear regulatory factors (NR), extracellular matrix (ECM), cell-surface (CS) molecules, protein manufacture (PM), protein degradation (PD), cell signaling (SI), blood specific factors muscle, and endothelial cell factors.

Transgenic Animals

The present invention also relates to transgenic animals comprising differentially-expressed genes as set forth in Table 1, and their homologs in other species. Such genes, as discussed in more detail below, include, but are not limited to, functionally-disrupted genes, mutated genes, ectopically or selectively-expressed genes, inducible or regulatable genes, etc. These transgenic animals can be produced according to any suitable technique or method, including homologous recombination, mutagenesis (e.g., ENU, Rathkolb et al., Exp. Physiol., 85(6):635-644, 2000), and the tetracycline-regulated gene expression system (e.g., U.S. Pat. No. 6,242,667). The term “gene” as used herein includes any part of a gene, i.e., regulatory sequences, promoters, enhancers, exons, introns, coding sequences, etc. The nucleic acid present in the construct or transgene can be naturally-occurring wild-type, polymorphic, or mutated. Transgenic animals thus produced can have phenotypes associated with defective angiongensis, e.g., excessive or insufficient angiogenesis.

Along these lines, polynucleotides of the present invention can be used to create transgenic animals, e.g. a non-human animal, comprising at least one cell whose genome comprises a functional disruption of a gene selected from Table 1, or a homolog thereof. By the phrases “functional disruption” or “functionally disrupted,” it is meant that the gene does not express a biologically-active product. It can be substantially deficient in at least one functional activity coded for by the gene. Expression of a polypeptide can be substantially absent, i.e., essentially undetectable amounts are made. However, polypeptide can also be made, but which is deficient in activity, e.g., where only an amino-terminal portion of the gene product is produced.

The transgenic animal can comprise one or more cells. When substantially all its cells contain the engineered gene, it can be referred to as a transgenic animal “whose genome comprises” the engineered gene. This indicates that the endogenous gene loci of the animal has been modified and substantially all cells contain such modification.

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the gene, etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. A transgenic animal which comprises the functional disruption can also be referred to as a “knock-out” animal, since the biological activity of its gene has been “knocked-out.” Knock-outs can be homozygous or heterozygous.

For creating functional disrupted genes, and other gene mutations, homologous recombination technology is of special interest since it allows specific regions of the genome to be targeted. Using homologous recombination methods, genes can be specifically-inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, a gene locus can be disrupted in mouse ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into an exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525.

A transgenic animal, or animal cell, lacking one or more functional genes can be useful in a variety of applications, including, as an animal model for angiogenic diseases (i.e., diseases associated with abnormal, excessive, or insufficient angiogenesis), for drug screening assays, and any of the utilities mentioned in any issued U.S. Patent on transgenic animals, including, U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824.

The present invention also relates to non-human, transgenic animal whose genome comprises recombinant gene selected from Table 1 operatively linked to an expression control sequence effective to express said coding sequence, e.g., in tissues capable of forming blood vessels. Such a transgenic animal can also be referred to as a “knock-in” animal since an exogenous gene has been introduced, stably, into its genome.

A recombinant nucleic acid refers to a nucleic acid which has been introduced into a target host cell and optionally modified, such as cells derived from animals, plants, bacteria, yeast, etc. A recombinant nucleic acid (or gene) includes completely synthetic nucleic acid sequences, semi-synthetic nucleic acid sequences, sequences derived from natural sources, and chimeras thereof. “Operable linkage” has the meaning used through the specification, i.e., placed in a functional relationship with another nucleic acid. When a gene is operably linked to an expression control sequence, as explained above, it indicates that the gene (e.g., coding sequence) is joined to the expression control sequence (e.g., promoter) in such a way that facilitates transcription and translation of the coding sequence. As described above, the phrase “genome” indicates that the genome of the cell has been modified. In this case, the recombinant gene has been stably integrated into the genome of the animal. The nucleic acid coding sequence is in operable linkage with the expression control sequence can also be referred to as a construct or transgene.

Any expression control sequence can be used depending on the purpose. For instance, if selective expression is desired, then expression control sequences which limit its expression can be selected. These include, e.g., tissue or cell-specific promoters, introns, enhancers, etc. For various methods of cell and tissue-specific expression, see, e.g., U.S. Pat. Nos. 6,215,040, 6,210,736, and 6,153,427. These also include the endogenous promoter, i.e., the coding sequence can be operably linked to its own promoter. Inducible and regulatable promoters can also be utilized.

The present invention also relates to a transgenic animal which contains a functionally disrupted and a transgene stably integrated into the animals genome. Such an animal can be constructed using combinations any of the above- and below-mentioned methods. Such animals have any of the aforementioned uses, including permitting the knock-out of the normal gene and its replacement with a mutated gene. Such a transgene can be integrated at the endogenous gene locus so that the functional disruption and “knock-in” are carried out in the same step.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet., 20:465-499, 1986; Askew et al., Mol. Cell. Bio., 13:4115-4124, 1993; Games et al. Nature, 373:523-527, 1995; Valancius and Smithies, Mol. Cell. Bio., 11: 1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993; Cibelli et al., Science, 280:1256-1258, 1998. For guidance on recombinase excision systems, see, e.g., U.S. Pat. Nos. 5,626,159, 5,527,695, and 5,434,066. See also, Orban, P.C., et al., “Tissue- and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at loxP-Containing DNA sequences placed into the mammalian genome,” Polynucleotides Research, 17(1):147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100: 169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-345, 1985), sheep (Hammer et al., Nature, 315:343-345, 1985), cattle, rat, or primate. See also, e.g., Church, 1987, Trends in Biotech. 5:13-19; Clark et al., Trends in Biotech. 5:20-24, 1987); and DePamphilis et al., BioTechniques, 6:662-680, 1988. Transgenic animals can be produced by the methods described in U.S. Pat. No. 5,994,618, and utilized for any of the utilities described therein.

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving gene sequences from a computer-readable medium, comprising, one or more of the following steps in any effective order, e.g., selecting a cell or gene expression profile, e.g., a profile that specifies that said gene is differentially expressed in cells capable of forming blood vessels, and retrieving said differentially expressed gene sequences, where the gene sequences consist of the genes selected from Table 1. The query can demand that said genes show sustained expression, transient expression, are only expressed at certain time point (e.g., 1, hr, 8, hr, or 24 hr), are associated with functional tubes (e.g., genes transiently expressed at 24-hours), etc.

A “gene expression profile” means the list of tissues, cells, etc., in which a defined gene is expressed (i.e, transcribed and/or translated). A “cell expression profile” means the genes which are expressed in the particular cell type. The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene or cell expression profile,” it is meant that a user decides what type of gene or cell expression pattern he is interested in retrieving, e.g., he may require that the gene is differentially expressed in a tissue, or he may require that the gene is not expressed in blood, but must be expressed in angiogenic forming tissues. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data. The database is information that has been stored in an appropriate storage medium, having a suitable computer-readable format. Once results are retrieved, they can be displayed in any suitable format, such as HTML.

For instance, the user may be interested in identifying genes that are differentially expressed in angiogenic forming tissue. He may not care whether small amounts of expression occur in other tissues, as long as such genes are not expressed in peripheral blood lymphocytes. A query is formed by the user to retrieve the set of genes from the database having the desired gene or cell expression profile. Once the query is inputted into the system, a search algorithm is used to interrogate the database, and retrieve results.

Advertising, Licensing, etc., Methods

The present invention also relates to methods of advertising, licensing, selling, purchasing, brokering, etc., genes, polynucleotides, specific-binding partners, antibodies, etc., of the present invention. Methods can comprises, e.g., displaying a gene selected from Table 1, polypeptides and specific binding partners thereof., in a printed or computer-readable medium (e.g., on the Web or Internet), accepting an offer to purchase said gene, polypeptide, or antibody.

Other

A polynucleotide, probe, polypeptide, antibody, specific-binding partner, etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment. A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the primary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

The topic headings set forth above are meant as guidance where certain information can be found in the application, but are not intended to be the only source in the application where information on such topic can be found.

Reference Materials

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fullest extent. The preceding specific embodiments are, therefore, to be construed as merely illustrative, and not limiting the remainder of the disclosure in any way whatsoever. The entire disclosure of all applications, patents and publications, cited above are hereby incorporated in their entirety, including U.S. Provisional Application No. 60/328,395, filed Oct. 12, 2002.

TABLE 1

| Gene identifier | | | Gene description |
|---|---|---|---|
| 210 | ANC0394D | D1SH | 12833637\|dbj\|AK003150.1\|AK003150 Mus musculus 18 days embryo cDNA, |
| | | | RIKEN full-length enriched |
| 210 | ANC0394D | D1SH | 179595\|J03464.1\|HUMC1A2 Human collagen alpha-2 type I mRNA, complete cds, clone |
| 248 | ANA1069D | D1SH | 12667797\|ref\|NM_022984.1\| Mus musculus resistin (Rstn) |
| 248 | ANA1069D | D1SH | AF323081 Homo sapiens resistin mRNA, complete cds |
| 529 | ANC3833D | D1SH | 12832312\|dbj\|AK002376.1\|AK002376 Mus musculus adult male kidney cDNA, |
| | | | RIKEN full-length enriched library, clone:0610009D10, full insert sequence |
| 529 | ANC3833D | D1SH | 3641297\|AF087135.1\|AF087135 Homo sapiens FIFO-type ATPase subunit d mRNA, |
| | | | nuclear gene encoding mitochondrial protein, complete cds |
| 212 | ANC0354D | D1SH | 1903415\|gb\|U76112.1\|MMU76112 Mus musculus translation represser NAT1 mRNA |
| | | | (used j), EST gb\|AA139483.1\|AA139483 mq86d01.r1 Stratagene mouse melanoma |
| | | | (#937312) (used F) |
| 212 | ANC0354D | D1SH | 1857236\|U73824.1\|HSU73824 Human p97 mRNA, complete cds |
| 18 | AN010745D | D1SL | 7305440\|ref\|NM_013762.1\| Mus musculus ribosomal protein L3 (Rpl3), mRNA |
| 18 | AN010745D | D1SL | 313658\|X73460.1\|HSRPL3A H.sapiens mRNA for ribosomal protein L3 |
| 25 | AN010563D | D1SL | 6678649\|ref\|NM_008477.1\| Mus musculus kinectin 1 (Ktn1), mRNA. |
| | | | EST: 2081547\|gb\|AA420267.1\|AA420267 vf50h03.r1 Soares mouse |
| | | | NbMH Mus musculus cDNA clone IMAGE:847253 (plus/plus) |
| 25 | AN010563D | D1SL | 4826813\|NM_004986.1\| Homo sapiens kinectin 1 (kinesin receptor) (KTN1), mRNA |
| 137 | AN021015D | D1SL | 9581820\|emb\|AJ278733.1\|MMU278733 Mus musculus partial mRNA for myosin heavy chain IIB |
| 137 | AN021015D | D1SL | 5814402\|AF111783.2\|AF111783 Homo sapiens myosin heavy chain IIb mRNA, complete cds |
| 164 | AN011867 | D1SL | 6678833\|ref\|NM_008569.1\| Mus musculus meiotic check point regulator (Mcpr), mRNA |
| 164 | AN011867 | D1SL | 12056970\|NM_022662.1\| Homo sapiens meiotic checkpoint regulator (MCPR), mRNA |
| 211 | ANC0356D | D1SL | 6671548\|ref\|NM_007453.1\| Mus musculus peroxiredoxin 5 (Prdx5) |
| 211 | ANC0356D | D1SL | D14662.1\|HUMORF06 Human mRNA for KIAA0106 gene, complete cds |
| 219 | ANC0575D | D1SL | 6678076\|ref\|NM_009242.1\| Mus musculus secreted acidic cysteine rich glycoprotein |
| 219 | ANC0575D | D1SL | 338312\|J03040.1\|HUMSPARC Human SPARC/osteonectin mRNA, complete cds |
| 221 | ANC0533D | D1SL | NM_019946.1\| Mus musculus microsomal glutathione S-transferase (Gst) |
| 221 | ANC0533D | D1SL | 183655\|J03746.1\|HUMGST Human glutathione S-transferase mRNA, complete cds |
| 265 | ANG1375D | D1SL | 6755255\|ref\|NM_011224.1 Mus musculus muscle glycogen phosphorylase |
| 265 | ANG1375D | D1SL | 5032008\|NM_005609.1\| Homo sapiens phosphorylase, glycogen; muscle |
| 319 | AN121219D | D1SL | 53988\|emb\|X00525.1\|MMRNA02 Mouse 28S ribosomal RNA |
| 319 | AN121219D | D1SL | 337381\|M11167.1\|HUMRGM Human 28S ribosomal RNA gene |
| 414 | AN101296D | D1SL | 13938071\|gb\|BC007152.1\|BC007152 Mus musculus, eukaryotic translation elongation |
| | | | factor 2, clone. EST: 12089106\|gb\|BF784070.1\|BF784070 602110020F1 |
| | | | NCI_CGAP_Kid14 Mus musculus cDNA clone IMAGE:4238261 (plus/plus) |
| 414 | AN101296D | D1SL | 31105\|X51466.1\|HSEF2 Human mRNA for elongation factor 2 |
| 438 | ANG0154D | D1SL | 6678931\|ref\|NM_008625.1\| Mus musculus mannose receptor, C type 1 (Mrc1), |
| | | | mRNA. EST: 6632659\|gb\|AW259678.1\|AW259678 uq40a02.x1 NCI_CGAP_Mam5 |
| | | | Mus musculus cDNA clone IMAGE:2811818 3′ (plus/plus) |
| 438 | ANG0154D | D1SL | 188675\|J05550.1\|HUMMRA Human mannose receptor mRNA, complete cds |
| 455 | ANC2156D | D1SL | 12847006\|dbj\|AK011102.1\|AK011102 Mus musculus 13 days embryo liver cDNA, |
| | | | RIKEN full-length enriched |
| 455 | ANC2156D | D1SL | 13788565\|NM_000518.3\| Homo sapiens hemoglobin, beta (HBB), mRNA |
| 459 | ANA0432D | D1SL | 13270470\|ref\|NM_024427.1\| Mus musculus alpha tropomyosin (Tpml), mRNA |
| 459 | ANA0432D | D1SL | 14752618\|XM_017091.1\| Homo sapiens tropomyosin 1 (alpha) (TPM1), mRNA |
| 532 | ANG3917D | D1SL | 13277848\|gb\|BC003804.1\|BC003804 Mus musculus, interferon-induced protein with |
| | | | tetratricopeptide |
| 532 | ANG3917D | D1SL | 3719293\|AF083470.1\|AF083470 Homo sapiens interferon induced tetratricopeptide |
| | | | protein IFI60 (IFIT4) mRNA, complete cds |
| 539 | ANA1543Da | D1SL | 13096996\|gb\|BC003290.1\|BC003290 Mus musculus, cyclin I, clone MGC:5636, mRNA, |
| | | | complete cds |
| 539 | ANA1543Da | D1SL | 7259481\|AF135162.1\|AF135162 Homo sapiens cyclin I (CYC1) mRNA, complete cds |
| 505 | ANG2814D | D1SL | M74773.1 GI:598330, Mus musculus brain beta spectrin (Spnb-2) mRNA, complete cds |
| 505 | ANG2814D | D1SL | 1805279\|U83867.1\|HSU83867 Human alpha II spectrin mRNA, complete cds |
| 261 | ANC1349D | D1TH | 12700621\|gb\|AY012114.1\| Mus musculus 12S ribosomal RNA gene, partial sequence; |
| | | | and tRNA-Val |
| 261 | ANC1349D | D1TH | Human mitochondrial DNA |
| 264 | ANG1377D | D1TH | 12837703\|dbj\|AK005267.1\|AK005267 Mus musculus adult male cerebellum cDNA, |
| | | | RIKEN full-length enriched |
| 264 | ANG1377D | D1TH | 2828146\|AF042384.1\|AF042384 Homo sapiens BC-2 protein mRNA, complete cds |
| 313 | AN051137D | D1TH | 12858615\|dbj\|AK018744.1\|AK018744 Mus musculus adult male kidney cDNA, |
| | | | RIKEN full-length |
| 313 | AN051137D | D1TH | 31192\|X62320.1\|HSEPIT1 H.sapiens mRNA for epithelin 1 and 2 |
| 360 | AN090841D | D1TH | 6678370\|ref\|NM_009394.1\| Mus musculus troponin C, fast skeletal (Tncs), mRNA |
| 360 | AN090841D | D1TH | 36728\|X07898.1\|HSTC2 Human mRNA for fast skeletal troponin C |
| 502 | ANA2891D | D1TH | 52850\|emb\|X16074.1\|MML34GBL Murine mRNA for L-34 galactoside-binding lectin |
| 502 | ANA2891D | D1TH | 179530\|M57710.1\|HUMBPIGE Human IgE-binding protein (epsilon-BP) mRNA, complete cds |
| 513 | ANC3422D | D1TH | 13605630\|gb\|AF361435.1 AF361435 Mus musculus neuronal development-associated |
| | | | protein 7 (Ndap7) mRNA |
| 513 | ANC3422D | D1TH | 14456614\|AB057724.1\|AB057724 Homo sapiens PIG-T mRNA for phosphatidyl inositol |
| | | | glycan class T, complete cds |
| 177 | AN060188D | D1TL | 12805174\|gb\|BC002046.1\|BC002046 Mus musculus, ephrin A1, clone MGC:6040, mRNA, |
| | | | complete cds |
| 177 | AN060188D | D1TL | 12805174\|BC002046.1\|BC002046 Mus musculus, ephrin A1, clone MGC:6040, mRNA, |
| | | | complete cds |
| 202 | AN070631D | D1TL | 199279\|gb\|M17440.1\|MUSMHC4H2S Mus musculus complement component C4A (C4A) gene, |
| | | | complete cds |
| 202 | AN070631D | D1TL | 13645583\|ref\|XP_004199.2 complement component 4A preproprotein (Homo sapiens) |
| 215 | ANG0383D | D1TL | 52848\|emb\|X06407.1\|MML21KD1 Mouse mRNA for 21 kd polypeptide under translational |
| | | | control |
| 215 | ANG0383D | D1TL | 37495\|X16064.1\|HSTUMP Human mRNA for translationally controlled tumor protein |
| 231 | ANC0726D | D1TL | 6678917\|ref\|NM_008618.1\| Mus musculus malate dehydrogenase, soluble (Mor2) |
| 231 | ANC0726D | D1TL | 1255603\|D55654.1 HUMCMD Human mRNA for cytosolic malate dehydrogenase, complete cds |
| 240 | ANC0943D | D1TL | 13173472\|ref\|NM_011170.1\| Mus musculus prion protein (Prnp), mRNA |
| 240 | ANC0943D | D1TL | 190467\|M13899.1\|HUMPRP Human prion protein (PrP) mRNA, complete cds |
| 250 | ANC1059D | D1TL | 12835788\|dbj\|AK004547.1\|AK004547 Mus musculus adult male lung cDNA, |
| | | | RIKEN full-length enriched |
| 250 | ANC1059D | D1TL | 1854034\|U86753.1\|HSU86753 Human Cdc5-related protein (PCDC5RP) mRNA, complete cds |
| 283 | AN061692D | D1TL | BC005704, 13543051 Mus musculus, Similar to hypothetical protein |
| 283 | AN061692D | D1TL | 7706155\|NM_016618.1\| Homo sapiens hypothetical protein (LOC51315), mRNA |
| 289 | AN050839D | D1TL | 50674\|emb\|X65157.1\|MMDES M.musculus mRNA for desmoyokin, partial |
| 289 | AN050839D | D1TL | 178280\|M80902.1\|HUMAHNAK Human AHNAK nucleoprotein mRNA, 5′ end |
| 295 | AN050938D | D1TL | 12659085\|gb\|AF318301.1\|AF318301 Mus musculus CGI-74-like SR-rich protein mRNA, |
| | | | complete cds |
| 295 | AN050938D | D1TL | AF151832 Homo sapiens CGI-74 protein mRNA, complete cds |
| 298 | AN051072D | D1TL | 12837800\|dbj\|AK005328.1\|AK005328 Mus musculus adult male cerebellum cDNA, |
| | | | RIKEN full-length enriched library, clone:1500031N16, full insert sequence |
| 298 | AN051072D | D1TL | X79865.1\|HSMRP17 H.sapiens Mrp17 mRNA |
| 314 | AN061190D | D1TL | 13529649\|gb\|BC005533.1\|BC005533 Mus musculus, Similar to ATP citrate lyase, clone |
| | | | IMAGE:3496817. EST: 10750811\|gb\|BF019479.1\|BF019479 ux10f12.y1 Soares_thymus_2NbMT |
| | | | Mus musculus cDNA clone (plus/plus) |
| 314 | AN061190D | D1TL | 603073\|U18197.1\|HSU18197 Human ATP:citrate lyase mRNA, complete cds |
| 335 | AN051871D | D1TL | 7656986f\|ref\|NM_015734.1\| Mus musculus procollagen, type V, alpha 1 (Col5a1), mRNA |
| 335 | AN051871D | D1TL | 189519\|M76729.1\|HUMPA1V Human pro-alpha-1 (V) collagen mRNA, complete cds |
| 345 | AN052038D | D1TL | 6677768\|ref\|NM_009076.1\| Mus musculus ribosomal protein L12 (Rp112), mRNA |
| 345 | AN052038D | D1TL | BC008230 Homo sapiens, ribosomal protein L12 |
| 353 | AN080627D | D1TL | 6755900\|ref\|NM_011653.1\| Mus musculus tubulin alpha 1 (Tuba1), mRNA |
| 353 | AN080627D | D1TL | 4929133\|AF141347.1\|AF141347 Homo sapiens hum-a-tub2 alpha-tubulin mRNA, complete cds |
| 437 | ANG0165D | D1TL | 6754853\|ref\|NM_010917.1\| Mus musculus nidogen 1 (Nid1), mRNA |
| 437 | ANG0165D | D1TL | 189208\|M30269.1\|HUMNID Human nidogen mRNA, complete cds |
| 449 | ANC1938D | D1TL | 10048445\|ref\|NM_020509.1\| Mus musculus resistin like alpha (Retnla), mRNA |
| 449 | ANC1938D | D1TL | AF323084 Homo sapiens resistin-like molecule beta mRNA, complete cds |
| 454 | ANC2081D | D1TL | 13625177\|gb\|AF251058.1\|AF251058 Homo sapiens clone 2 thrombospondin mRNA, |
| | | | complete cds 56 bp??? |
| 454 | ANC2081D | D1TL | 13625177\|AF251058.1\|AF251058 Homo sapiens clone 2 thrombospondin mRNA, complete cds |
| 485 | ANG2612D | D1TL | 6671588\|ref\|NM_007504.1\| Mus musculus ATPase, Ca++ transporting, cardiac muscle, |
| | | | fast twitch |
| 485 | ANG2612D | D1TL | 10835219\|NM_004320.1\| Homo sapiens ATPase, Ca++ transporting, cardiac muscle, |
| | | | fast twitch 1(ATP2A1), mRNA |
| 514 | ANG3444D | D1TL | 53990\|emb\|X00686.1\|MMRNA18 Mouse gene for 18S rRNA. EST: |
| | | | P/P 12564320\|gb\|BG081752.1\|BG081752 H3068F10-5 NIA Mouse |
| | | | 15K cDNA Clone Set Mus musculus cDNA clone |
| 514 | ANG3444D | D1TL | AF225896 Homo sapiens tensin mRNA, complete cds |
| 520 | ANA3611D | D1TL | 6753667\|ref\|NM_010071.1\| Mus musculus downstream of tyrosine kinase 2 (Dok2), mRNA. |
| | | | EST p/p: 3175820\|gb\|AA990456.1\|AA990456 ua59h05.s1 Soares_thymus_2NbMT |
| | | | Mus musculus cDNA clone |
| 520 | ANA3611D | D1TL | 3043918\|AF034970.1\|AF034970 Homo sapiens docking protein (DOK-2) mRNA, complete cds |
| 521 | ANA3643D | D1TL | 6273571\|emb\|AJ246002.1\|MMU246002 Mus musculus mRNA for spastin protein orthologue |
| | | | (Spast gene) |
| 521 | ANA3643D | D1TL | 6273490\|AJ246001.1\|HSA246001 Homo sapiens mRNA for spastin protein (Spast gene) |
| 525 | ANA3752D | D1TL | 7305492\|ref\|NM_013929.1\| Mus musculus Cd27 binding protein (Hindu God of destruction) |
| | | | (Siva-pending), mRNA |
| 525 | ANA3752D | D1TL | U82938.1\|HSU82938 Human CD27BP (Siva) mRNA, complete cds |
| 530 | ANA3958D | D1TL | 3986763\|gb\|AF109906.1\|MMHC310M6 Mus musculus MHC class III region RD gene, |
| | | | partial cds; complete cds; G7A gene, partial cds; and unknown genes. |
| 530 | ANA3958D | D1TL | 14782421\|XM_004195.2\| Homo sapiens RD RNA-binding protein (RDBP), mRNA |
| 537 | ANA0232D | D1TL | 13435743\|gb\|BC004731.1\|BC004731 Mus musculus, integral membrane protein 2 B, |
| | | | clone MGC:6390, mRNA,complete cds |
| 537 | ANA0232D | D1TL | 12653560\|BC000554.1\|BC000554 Homo sapiens, Similar to integral membrane protein 2B, |
| | | | clone |
| 541 | ANC0120D | D1TL | 13643757\|ref\|XM_016250.1\| Homo sapiens titin (TTN), mRNA |
| 541 | ANC0120D | D1TL | 14732031\|XM_016250.1\| Homo sapiens titin (TTN), mRNA |
| 198 | AN050733D | D24H | 13277860\|gb\|BC003809.1\|BC003809 Mus musculus, Similar to t-complex protein 1, |
| | | | clone MGC:6094, mRNA |
| 198 | AN050733D | D24H | 12653758\|BC000665.1\|BC000665 Homo sapiens, t-complex 1, clone MGC:2234, mRNA, |
| | | | complete cds |
| 76 | ANA0966D | D24L | 12843389\|dbj\|AK008914.1\|AK008914 Mus musculus adult male stomach cDNA, |
| | | | RIKEN full-length enriched |
| 76 | ANA0966D | D24L | 14290493\|BC009015.1\|BC009015 Homo sapiens, serine (or cysteine) proteinase |
| | | | inhibitor, clade B (ovalbumin), member 1, clone MGC:9340 IMAGE:3456154, mRNA, |
| | | | complete cds |
| 176 | AN060182D | D24L | 12805364\|gb\|BC002152.1\|BC002152 Mus musculus, Similar to mitochondrial carrier |
| | | | homolog 2, clone |
| 176 | AN060182D | D24L | AF176008 Homo sapiens mitochondrial carrier homolog 2 mRNA, complete cds |
| 192 | AN060440D | D24L | AF343079 Mus musculus TOB3 mRNA, complete cds |
| 192 | AN060440D | D24L | 13752410\|AF343078.1\|AF343078 Homo sapiens TOB3 mRNA, complete cds |
| 129 | AN040829D | D8SH | 12805086\|gb\|BC002000.1\|BC002000 Mus musculus, opioid receptor, sigma 1, |
| | | | clone MGC:5760, mRNA |
| 129 | AN040829D | D8SH | 1906590\|U75283.1\|HSU75283 Human sigma receptor mRNA, complete cds |
| 534 | ANG3965D | D8SH | 205965\|gb\|J05206.1\|RATPAI1AA Rat plasminogen activator inhibitor-1. |
| | | | EST p/p: 8878242\|dbj\|BB213289.1\|BB213289 BB213289 RIKEN full-length |
| | | | enriched, adult male aorta and vein Mus |
| 534 | ANG3965D | D8SH | 189541\|M16006.1\|HUMPAI Human plasminogen activator inhibitor-1 (PAI-1) |
| | | | mRNA, complete cds |
| 38 | AN010386D | D8SL | 7657428\|reflNM_0 15784.1 1 Mus musculus osteoblast specific factor 2 |
| | | | (fasciclin I-like) (Osf2-pending), mRNA. EST: 5125957\|gb\|AI747693.1\|AI747693 |
| | | | u121a11.x1 Sugano mouse embryo mewa Mus musculus cDNA clone (plus/minus) |
| 38 | AN010386D | D8SL | D13666.1\|HUMOSF2OS Homo sapiens osf-2 mRNA for osteoblast specific factor 2 |
| 39 | AN010357D | D8SL | 6680839\|ref\|NM_007594.1\| Mus musculus calumenin (Calu), mRNA |
| 39 | AN010357D | D8SL | 3153208\|AF013759.1\|AF013759 Homo sapiens calumein (Calu) mRNA, complete cds |
| 86 | ANC102kD | D8SL | 13277944\|gb\|BC003839.1\|BC003839 Mus musculus, myeloid cell leukemia sequence 1, |
| | | | clone MGC:6351 |
| 86 | ANC102kD | D8SL | 4235636\|AF118124.1\|AF118124 Homo sapiens myeloid cell leukemia sequence 1 (MCL1) mRNA, |
| | | | complete cds |
| 144 | AN011135Da | D8SL | 7657563\|ref\|NM_012059.2 Mus musculus SH3 domain protein D19 (Sh3d19), mRNA |
| 144 | AN011135Da | D8SL | 6453460\|AL133047.1\|HSM801318 Homo sapiens mRNA; cDNA DKFZp434D0215 |
| | | | (from clone DKFZp434D0215); partial cds |
| 238 | ANC0983D | D8TH | AK003930.1\|AK003930 Mus musculus 18 days embryo cDNA, RIKEN full-length |
| | | | enriched library; Est: >gb\|AA645845.1\|AA645845 vs31b12.r1 Stratagene |
| | | | mouse Tcell 937311 Mus musculus cDNA clone (used C) |
| 238 | ANC0983D | D8TH | 188621\|M93056.1\|HUMMNEI Human mononcyte/neutrophil elastase inhibitor mRNA sequence |
| 121 | AN040621U | U1SH | 6754455\|ref\|NM_010637.1\| Mus musculus Kruppel-like factor 4 (gut) (K1f4) |
| 121 | AN040621tT | U1SH | 5353532\|AF105036.1\|AF105036 Homo sapiens zinc finger transcription factor GKLF mRNA, |
| | | | complete cds |
| 123 | AN040743U | U1SH | 12852319\|dbj\|AK014456.1\|AK014456 Mus musculus 13 days embryo cDNA, |
| | | | RIKEN full-length enriched library. |
| 123 | AN040743U | U1SH | 13194723\|AF326966.1\|AF326966 Homo sapiens cytokine-like nuclear factor n-pac mRNA, |
| | | | complete cds |
| 184 | AN060390U | U1SH | 10946617\|ref\|NM_021314.1\| Mus musculus transforming, acidic coiled-coil |
| | | | containing protein 2 |
| 184 | AN060390U | U1SH | 11096459\|AF095791.2\|AF095791 Homo sapiens TACC2 protein (TACC2) mRNA, complete cds |
| 296 | AN060942U | U1SH | 12837615\|dbj\|AK005212.1\|AK005212 Mus musculus adult male cerebellum cDNA, |
| | | | RIKEN full-length enriched library, clone:1500011112, full insert sequence |
| 296 | AN060942U | U1SH | 7021874\|AK000913.1\|AK000913 Homo sapiens cDNA FLJ10051 fis, clone HEMBA1001281 |
| 381 | AN091446U | U1SH | 9845264\|ref\|NM_019883.1\| Mus musculus ubiquitin A-52 residue ribosomal protein fusion |
| 381 | AN091446U | U1SH | 37564\|X56998.1\|HSUBA52A Human UbA52 adrenal mRNA for ubiquitin-52 amino acid fusion protein |
| 384 | AN091944U | U1SH | 12005325\|gb\|AF239176.1\|AF239176 Mus musculus pyruvate dehydrogenase kinase 4 (Pdk4) gene, |
| | | | complete cds |
| 384 | AN091944U | U1SH | 4505693\|ref\|NP_002603.1\| pyruvate dehydrogenase kinase, isoenzyme 4 [Homo sapiens] |
| 17 | AN010756U | U1SL | 220617\|dbj\|D10061.1\|MUSTOPIA Mus musculus mRNA for DNA topoisomerase I, complete cds |
| 17 | AN010756U | U1SL | 339805\|J03250.1\|HUMTOPI Human topoisomerase I mRNA, complete cds |
| 58 | AN030634U | U1SL | 9790202\|ref\|NM_019642.1\| Mus musculus ribophorin II (Rpn2), mRNA |
| 58 | AN030634U | U1SL | 13097707\|BC003560.1\|BC003560 Homo sapiens, ribophorin II, clone MGC:1817 IMAGE:3546673, mRNA |
| 80 | ANC0911aU | U1SL | 193329\|gb\|M18194.1\|MUSFN Mouse fibronectin (FN) mRNA |
| 80 | ANC0911aU | U1SL | 31396\|emb\|X02761.1\|HSFIB1 Human mRNA for fibronectin (FN precursor) |
| 100 | AN020298U | U1SL | 3805816\|emb\|Y17159.1\|MMU17159 Mus musculus mRNA for SH2-containing leukocyte protein 65 |
| 100 | AN020298U | U1SL | 3406748\|AF068180.1\|AF068180 Homo sapiens B cell linker protein BLNK mRNA, |
| | | | alternatively spliced, complete cds |
| 107 | AN010438U | U1SL | 6753337\|ref\|NM_009844.1 Mus musculus CD19 antigen (Cd19), mRNA |
| 107 | AN010438U | U1SL | 862622\|M28170.1\|HUMCSPC Human cell surface protein CD19 (CD 19) gene, complete cds |
| 122 | AN040763U | U1SL | 12847140\|dbj\|AK011184.1\|AK011184 Mus musculus 10 days embryo cDNA, |
| | | | RIKEN full-length enriched library |
| 122 | AN040763U | U1SL | 887359\|L40392.1\|HUMORFB Homo sapiens (clone S164) mRNA, 3′ end of cds |
| 148 | AN041191U | U1SL | 12836737\|dbj\|AK005067.1\|AK005067 Mus musculus adult male liver cDNA, |
| | | | RIKEN full-length enriched |
| 148 | AN041191U | U1SL | 12804476\|BC001646.1\|BC001646 Homo sapiens, clone MGC:2392, mRNA, complete cds |
| 162 | AN021769 | U1SL | 13699330\|gb\|AC091424.1\|AC091424 Mus musculus chromosome 11 clone MGS1-139O9, |
| | | | complete sequence. EST: p/p 7066595\|gb\|AW496305.1\|AW496305 up51h06.y1 |
| | | | Soares_mouse_NMGB_bcell Mus musculus cDNA clone |
| 162 | AN021769 | U1SL | 12654846\|BC001267.1\|BC001267 Homo sapiens, RAB5A, member RAS oncogene family, |
| | | | clone MGC:5048, mRNA |
| 200 | AN120648U | U1SL | 12848185\|dbj\|AK011820.1\|AK011820 Mus musculus 10 days embryo cDNA, |
| | | | RIKEN full-length enriched library |
| 200 | AN120648U | U1SL | 6164619\|AF129535.1\|AF129535 Homo sapiens F-box protein Fbx5 (FBX5) mRNA, complete cds |
| 214 | ANG0313U | U1SL | 11275297\|dbj\|AB020974.1\|AB020974 Mus musculus mRNA for MAIL, complete cds |
| 214 | ANG0313U | U1SL | 13516830\|AB037925.1\|AB037925 Homo sapiens MAIL mRNA, complete cds. |
| | | | Origene human EST: >PR1357_B08.g1. |
| 256 | ANC1234U | U1SL | 200769\|gb\|M85235.1\|MUSRP Mus musculus ribosomal protein mRNA, complete cds. |
| | | | EST: 5137256\|dbj\|AV051484.1\|AV051484 AV051484 Mus musculus pancreas C57BL/6J |
| | | | adult Mus musculus cDNA (plus/minus) |
| 256 | ANC1234U | U1SL | L16558.1\|HUMRPL7Y Human ribosomal protein L7 (RPL7) mRNA, complete cds |
| 309 | AN051171U | U1SL | 984937\|gb\|U13393.1\|MMU13393 Mus musculus delta proteasome subunit mRNA, complete cds. EST: |
| | | | 1493731\|gb\|AA027711.1\|AA027711 mi12f08.r1 Scares mouse p3NMF19.5 Mus musculus cDNA clone |
| | | | (plus/plus) |
| 309 | AN051171U | U1SL | 12654058\|BC000835.1\|BC000835 Homo sapiens, Similar to proteasome |
| | | | (prosome, macropain) subunit beta type 6, clone MGC:5169, mRNA, complete cds |
| 321 | AN071331U | U1SL | 194389\|gb\|J00448.1\|MUSIGCD15 Mouse germline IgD gene, DJC region: c-delta-h(hinge) exon |
| 321 | AN071331U | U1SL | 106368\|pir\|\|S17597 Ig delta chain (WIE) - human |
| 324 | AN051785U | U1SL | 12851000\|dbj\|AK013583.1\|AK013583 Mus musculus adult male hippocampus cDNA, |
| | | | RIKEN full-length enriched library, clone:2900024E01, full insert sequence |
| 324 | AN051785U | U1SL | 6630621\|AB029150.1\|AB029150 Homo sapiens mRNA for KRAB zinc finger protein HFB101L, complete cds |
| 348 | AN090247U | U1SL | 6753197\|ref\|NM_009760.1\| Mus musculus BCL2/adenovirus E1B 19 kDa-interacting protein 1, |
| | | | NIP3. EST: 10650678\|gb\|BE981505.1\|BE981505 UI-M-CG0p-bdc-h-11-O-UI.s1 NIH_BMAP_Ret4_S2 |
| | | | Mus musculus cDNA clone (plus/minus) |
| 348 | AN090247U | U1SL | 14424635\|BC009342.1\|BC009342 Homo sapiens, BCL2/adeno virus E1B 19kD-interacting |
| | | | protein 3, clone , mRNA, complete cds |
| 398 | AN100536U | U1SL | 6754255\|ref\|NM_010481.1\| Mus musculus heat shock protein, 74 kDa, A (Hspa9a), mRNA |
| 398 | AN100536U | U1SL | 12653414\|BC000478.1\|BC000478 Homo sapiens, heat shock 70kD protein 9B (mortalin-2), |
| | | | clone MGC:8684, mRNA, complete cds |
| 400 | AN100726U | U1SL | 6755926\|ref\|NM_011909.1\| Mus musculus ubiquitin specific protease 18 (Usp18), mRNA |
| 400 | AN100726U | U1SL | AF176642 Homo sapiens ubiquitin-specific protease ISG43 (ISG43) mRNA |
| 488 | ANC2744U | U1SL | 6678522\|ref\|NM_009481.1\| Mus musculus ubiquitin specific protease 9, X chromosome (Usp9x), |
| | | | mRNA. EST p/m: 12545663\|gb\|BG063012.1\|BG063012 H3003A11-3 NIA Mouse 15K cDNA Clone Set |
| | | | Mus musculus cDNA clone |
| 488 | ANC2744U | U1SL | 6007846\|gb\|AF000986.2\|HSAF000986 Homo sapiens chromosome Y ubiquitin specific protease 9 |
| | | | (USP9Y) mRNA |
| 516 | ANA3543U | U1SL | 10181207\|ref\|NM_020585.1\| Mus musculus hypothetical protein, MNCb-1213 (AB041568), mRNA |
| 516 | ANA3543U | U1SL | 10140848\|NM_016099.1\| Homo sapiens HSPC041 protein (LOC51125), mRNA |
| 527 | ANA3846Ua | U1SL | 6681188\|ref\|NM_007861.1\| Mus musculus dihydrolipoamide dehydrogenase (Dld), |
| | | | mRNA. EST p/m: 4726194\|gb\|AI647516.1\|AI647516 uk40f05.x1 Sugano mouse kidney |
| | | | mkia Mus musculus cDNA clone |
| 527 | ANA3846Ua | U1SL | 181574\|J03620.1\|HUMDLDH Human dihydrolipoamide dehydrogenase mRNA, complete cds |
| 531 | ANA3943U | U1SL | 2275009\|emb\|X81624.1\|MAC111 M.auratus mRNA for C11 protein (C11-1) |
| 531 | ANA3943U | U1SL | 14727068\|XM_003686.3\| Homo sapiens eukaryotic translation termination factor 1 (ETF1), mRNA |
| 91 | AN010141U | U1TH | NM_025379 Mus musculus cytochrome c oxidase subunit VIIb (Cox7b), mRNA |
| 91 | AN010141U | U1TH | 4502990\|NM_001866.1\| Homo sapiens cytochrome c oxidase subunit Vllb (COX7B), mRNA |
| 135 | AN011038U | U1TH | 511867\|gb\|M62470.1\|MUSTHREX22 Mus musculus thrombospondin (THBS1) gene, exon 22 and complete cds |
| 135 | AN011038U | U1TH | 14749307\|XM_007606.2\| Homo sapiens thrombospondin 1 (THBS1), mRNA |
| 187 | AN050444UA | U1TH | AK012290 Mus musculus 11 days embryo cDNA, RIKEN full-length enriched library |
| 187 | AN050444UA | U1TH | NM_014739,Homo sapiens KIAA0164 gene product (KIAA0164) |
| 232 | ANG0713U | U1TH | 9885277\|gb\|AF199491.1\|AF199491 Mus musculus NOCTURNIN (Noctumin) mRNA |
| 232 | ANG0713U | U1TH | 5924315\|AF183961.1 AF183961 Homo sapiens carbon catabolite repression 4 protein |
| 252 | ANG1115U | U1TH | 7106274\|ref\|NM_007788.1\| Mus musculus casein kinase II, alpha 1, related sequence 4 |
| 252 | ANG1115U | U1TH | 177993\|M55265.1\|HUMACKII Human casein kinase II alpha subunit mRNA, complete cds |
| 294 | AN050943U | U1TH | 13097524\|gb\|BC003489.1\|BC003489 Mus musculus, Similar to acidic protein rich in leucines, clone |
| 294 | AN050943U | U1TH | Y07569.1\|HSPHAPI2A H.sapiens mRNA for PHAPI2a protein |
| 312 | AN051143bU | U1TH | 9507096\|ref\|NM_019464.1\| Mus musculus SH3-domain GRB2-like B1 (endophilin) (Sh3glb1), mRNA |
| 312 | AN051143bU | U1TH | 4929590\|AF151819.1\|AF151819 Homo sapiens CGI-61 protein mRNA, complete cds |
| 346 | AN072052U | U1TH | 2720357\|gb\|AA710439.1\|AA710439 vt42e01.r1 Barstead mouse proximal colon MPLRB6 Mus musculus cDNA |
| 346 | AN072052U | U1TH | 2911115\|AB002803.1\|AB002803 Homo sapiens BACH1 mRNA, complete cds |
| 463 | ANG0485U | U1TH | 12805642\|gb\|BC002302.1\|BC002302 Mus musculus, clone IMAGE:3591001, mRNA, partial cds |
| 463 | ANG0485U | U1TH | 5689568\|AB029039.1\|AB029039 Homo sapiens mRNA for KIAA1116 protein, complete cds |
| 247 | ANA1083U | U1TH | 198954\|gb\|L04264.1\|MUSLYSOX01 Mus musculus (clones p11-5.4, p11-3.8 and |
| | | | p99-5) lysyl oxidasegene(putative) -used c |
| 247 | ANA1083U | U1TH | 14722557\|XM_003695.3\| Homo sapiens lysyl oxidase (LOX), mRNA |
| 156 | AN011538 | U1TL | 12484083\|gb\|AF313412.11 AF313412 Mus musculus putative small membrane |
| | | | protein NID67 mRNA, complete cds. |
| 156 | AND 11 538 | U1TL | 12484085\|AF313413.1\|AF313413 Homo sapiens putative small membrane protein NID67 mRNA, |
| | | | complete cds |
| 218 | ANC0590D | U1TL | 7106416\|ref\|NM_009210.1\| Mus musculus SWI/SNF related, matrix associated, actin dependent |
| 218 | ANC0590D | U1TL | 531195\|L34673.1\|HUMHIP116A Human ATPase, DNA-binding protein (HIP116) mRNA |
| 237 | ANC0911U | U1TL | 13195146\|gb\|AY027438.1\| Mus musculus HCH mRNA, complete cds |
| 237 | ANC0911U | U1TL | D86964.1\|D86964 Human mRNA for KIAA0209 gene, partial cds |
| 288 | AN050842U | U1TL | 13543180\|gb\|BC005758.1\|BC005758 Mus musculus, clone IMAGE:3601067, mRNA, partial cds EST: P/M |
| | | | 12577524\|gb\|BG094972.1\|BG094972 uu82d06.x1 Soares_mouse_NMGB_bcell Mus musculus cDNA clone |
| 288 | AN050842U | U1TL | 187280\|L03532.1\|HUMM4PRO Human M4 protein mRNA, complete cds |
| 307 | AN121091U | U1TL | 13399307\|ref\|NM_025846.1\| Mus musculus RIKEN cDNA 2610016H24 gene (2610016H24Rik), mRNA. EST: |
| | | | 4703235\|gb\|AI640126.1\|AI640126 ms73f09.y1 Soares mouse 3NbMS Mus musculus cDNA clone |
| | | | IMAGE:617225 (plus/plus) |
| 307 | AN121091U | U1TL | 190876\|M31468.1\|HUMRASAB Human ras-like protein mRNA, complete cds, clone TC21 |
| 332 | AN071744U | U1TL | 2555188\|gb\|AF027865.1\|MMMMH461 Mus musculus Major Histocompatibility Locus class II region |
| 332 | AN071744U | U1TL | 619805\|M25327.1\|HUMMHCDQ33 Homo sapiens MHC HLA-DBQ3 allele DQw3.3 mRNA, 5′ |
| 333 | AN071743U | U1TL | 6678823\|ref\|NM_008562.1\| Mus musculus myeloid cell leukemia sequence 1 (Mc11), mRNA |
| 333 | AN071743U | U1TL | AF118124 Homo sapiens myeloid cell leukemia sequence 1 (MCL1) mRNA |
| 382 | AN091734U | U1TL | 6681284\|ref\|NM_007913.1\| Mus musculus early growth response 1 (Egr1), mRNA |
| 382 | AN091734U | U1TL | 182262\|M62829.1\|HUMETR103 Human transcription factor ETR103 mRNA, complete cds |
| 387 | AN100135Ua | U1TL | 12861969\|dbj\|AK021173.1\|AK021173 Mus musculus ES cells cDNA, |
| | | | RIKEN full-length enriched library |
| 387 | AN100135Ua | U1TL | 3335135\|AF047440.1\|AF047440 Homo sapiens ribosomal protein L33-like protein mRNA, complete cds |
| 443 | ANC0232U | U1TL | 6680763\|ref\|NM_007520.1\| Mus musculus BTB and CNC homology 1 (Bach1), mRNA |
| 443 | ANC0232U | U1TL | NM_001186.1 Homo sapiens BTB and CNC homology 1, basic leucine zipper |
| 472 | ANA2033U | U1TL | 6680108\|ref\|NM_008176.1\| Mus musculus GRO1 oncogene (Gro1), mRNA |
| 472 | ANA2033U | U1TL | M57731.1\|HUMGROB Human gro-beta mRNA, complete cds |
| 483 | ANC2634U | U1TL | 12832641\|dbj\|AK002567.1\|AK002567 Mus musculus adult male kidney cDNA, |
| | | | RIKEN full-length enriched library, clone:0610011P06, full insert sequence. |
| | | | EST P/M: 4317467\|gb\|AI463437.1\|AI463437 uc45b12.x1 Soares_mammary_gland_NMLMG |
| | | | Mus musculus cDNA clone |
| 483 | ANC2634U | U1TL | 13937856\|BC007034.1\|BC007034 Homo sapiens, metallothionein 2A, clone MGC:12397, mRNA, |
| | | | complete cds |
| 494 | ANA3122U | U1TL | 6753877\|ref\|NM_010217.1\| Mus musculus fibroblast inducible secreted protein (Fisp12), mRNA |
| 494 | ANA3122U | U1TL | 180923\|M92934.1\|HUMCONGRO Human connective tissue growth factor, complete cds |
| 523 | ANC3659U | U1TL | 12805426\|gb\|BC002186.1\|BC002186 Mus musculus, Similar to ribosomal protein S2, clone MGC:7380 |
| 523 | ANC3659U | U1TL | 14043190\|BC007583.1\|BC007583 Homo sapiens, clone MGC:15572, mRNA, complete cds |
| 542 | ANC0214U | U1TL | 13097338\|gb\|BC003420.1\|BC003420 Mus musculus, DNA J protein, clone MG:6445, |
| | | | mRNA, complete cds. EST p/p: 12771487\|gb\|BG261567.1\|BG261567 602373442F1 |
| | | | NIH_MGC_94 Mus musculus cDNA clone |
| 542 | ANC0214U | U1TL | 3171907\|AJ001309.1\|HSH4DNAJ Homo sapiens mRNA for DnaJ protein |
| 565 | ANG3121Ua | U1TL | 13905001\|gb\|BC006783.1\|BC006783 Mus musculus, connective tissue growth factor, |
| | | | clone MGC:8122, mRNA. EST: 11272375\|gb\|BF322900.1\|BF322900 maa35f04.x1 |
| | | | NCI_CGAP_Brn63 Mus musculus cDNA clone (p/m). |
| 565 | ANG3121Ua | U1TL | M92934.1 HUMCONGRO Human connective tissue growth factor, complete cds |
| 571 | ANC3240U | U1TL | 199131\|gb\|K02236.1\|MUSMETII Mouse metallothionein II (MT-II) gene. EST: |
| | | | 12596220\|gb\|BG100903.1\|BG100903 uy16h10.y1 McCarrey Eddy spermatocytes |
| | | | Mus musculus cDNA clone, (p/m) |
| 571 | ANC3240U | U1TL | X97260.1\|HSMTISO2 H.sapiens mRNA for metallothionein isoform 2 |
| 322 | AN121327aU | U24H | 6671508\|ref\|NM_007393.1\| Mus musculus melanoma X-actin (Actx), mRNA |
| 322 | AN121327aU | U24H | 28251\|X00351.1\|HSAC07 Human mRNA for beta-actin |
| 334 | AN121758U | U24L | 202434\|gb\|M60419.1\|MUSYBOXDNA Mouse Y-box binding protein 1/DNA binding protein B mRNA, |
| | | | complete cds |
| 334 | AN121758U | U24L | 181485\|M24070.1\|HUMDBPB Human DNA-binding protein B (dbpB) gene, 3′ end |
| 340 | AN121899U | U24L | 470673\|gb\|U08020.1\|MMU08020 Mus musculus FVB/N collagen pro-alpha-1 type I chain mRNA, |
| | | | complete |
| 340 | AN121899U | U24L | 1418927\|Z74615.1\|HSPPA1ICO H.sapiens mRNA for prepro-alpha1(I) collagen |
| 97 | AN040124U | U8SH | 6680641\|ref\|NM_007403.1\| Mus musculus a disintegrin and metalloprotease domain 8 |
| | | | (Adam8), mRNA |
| 97 | AN040124U | U8SH | 14736113\|XM005675.2\| Homo sapiens a disintegrin and metalloproteinase domain 8 (ADAM8) |
| 124 | AN040723U | U8SH | 12833637\|dbj\|AK003150.1\|AK003150 Mus musculus 18 days embryo cDNA, |
| | | | RIKEN full-length enriched |
| 124 | AN040723U | U8SH | 1418929\|Z74616.1\|HSPPA2ICO H.sapiens mRNA for prepro-alpha2(I) collagen |
| 236 | ANG0725U | U8SH | 11275668\|gb\|AF225896.1\|AF225896 Homo sapiens tensin mRNA |
| 236 | ANG0725U | U8SH | 11275668\|AF225896.1\|AF225896 Homo sapiens tensin mRNA, complete cds |
| 24 | AN010511U | U8SL | 3132609\|gb\|AF062567.1\|AF062567 Mus musculus transcription factor Sp3 mRNA, partial cds |
| 24 | AN010511U | U8SL | 38417\|X68560.1\|HSSPR2 H.sapiens SPR-2 mRNA for GT box binding protein |
| 92 | AN020148U | U8SL | 437044\|emb\|X70032.1\|MMNEB M.musculus mRNA for nebulin |
| 92 | AN020148U | U8SL | 1205987\|U35636.1\|HSNEBUL1 Human nebulin mRNA, partial cds |
| 105 | AN020343U | U8SL | 12833152\|dbj\|AK002859.1\|AK002859 Mus musculus adult male kidney cDNA, |
| | | | RIKEN full-length library |
| 105 | AN020343U | U8SL | 455833\|S67156.1\|S67156 ASP = aspartoacylase [human, kidney, mRNA, 1435 nt] |
| Ill | AN040527U | U8SL | 12848608\|dbj\|AK012085.1\|AK012085 Mus musculus 10 days embryo cDNA, RIKEN full-length |
| 111 | AN040527U | U8SL | 307313\|M96954.1\|HUMNUCTIAR Homo sapiens nucleolysin TIAR mRNA, complete cds |
| 315 | AN071128U | U8SL | 9910485\|ref\|NM_019932.1\| Mus musculus platelet factor 4 (Pf4), mRNA |
| 315 | AN071128U | U8SL | 189850\|M25897.1\|HUMPF4A Human platelet factor 4 (PF4) mRNA, complete cds |
| 338 | AN071895U | U8SL | 13385627\|ref\|NM_026123.1\| Mus musculus RIKEN cDNA 1110002A21 gene (1110002A21Rik), mRNA |
| 338 | AN071895U | U8SL | 14721863\|XM_002492.3\| Homo sapiens DKFZP564G0222 protein (DKFZP564G0222), mRNA |
| 393 | AN100312U | U8SL | 13277683\|gb\|BC003746.1\|BC003746 Mus musculus, Similar to microspherule protein 1, |
| | | | clone MGC:5852. EST: 10977531\|gb\|BF138491.1\|BF138491 601782948F1 NCI_CGAP_Lu30 |
| | | | Mus musculus cDNA clone IMAGE:4011107 5′ (plus/plus) |
| 393 | AN100312U | U8SL | 2384716\|AF015308.1\|AF015308 Homo sapiens nucleolar protein (MSP58) mRNA, complete cds |
| 47 | AN010421U | U8TH | 53309\|emb\|X04417.1\|MMMYOGG2 M.musculus myoglobin gene exons 2-3. EST: |
| | | | 9572630\|dbj\|BB521172.1\|BB521172 RIKEN full-length enriched, 16 days neonate heart |
| | | | Mus musculus cDNA clone D830048I22 3′ (plus/plus) |
| 47 | AN010421U | U8TH | ref\|XM_009949.1\| Homo sapiens myoglobin (MB), mRNA |
| 119 | AN020621U | U8TH | 12841780\|dbj\|AK007918.1\|AK007918 Mus musculus 10 day old male pancreas cDNA, |
| | | | RIKEN full-length |
| 119 | AN020621U | U8TH | 425517\|S65761.1\|S65761 anti-colorectal carcinoma heavy chain = glycoprotein |
| | | | CANAG-50 specific IgG1 kappa [human, 19.9 hybridoma, antibody, mRNA |
| 120 | AN040627U | U8TH | 11437688\|ref\|XM_006281.1\| Homo sapiens gastric intrinsic factor (vitamin B synthesis) |
| | | | (GIF)??? |
| 120 | AN040627U | U8TH | 806639\|M63154.1\|HUMGIF Human intrinsic factor mRNA, complete cds |
| 143 | AN041068U | U8TH | 7949077\|ref\|NM_016754.1 Mus musculus myosin light chain 2 (Mlc2) |
| 143 | AN041068U | U8TH | AF363061 Homo sapiens myosin regulatory light chain 2 (MRLC2) mRNA, |
| 152 | AN011442 | U8TH | 10946747\|ref\|NM_021400.1\| Mus musculus proteoglycan 3 (megakaryocyte stimulating factor, |
| 152 | ANO 11442 | U8TH | 14730483\|XM_001738.2\| Homo sapiens proteoglycan 4, (megakaryocyte stimulating |
| | | | factor articular superficial zone protein) (PRG4), mRNA |
| 197 | AN050727U | U8TH | 12835429\|dbj\|AK004296.1\|AK004296 Mus musculus 18 days embryo cDNA, |
| | | | RIKEN full-length enriched library |
| 197 | AN050727U | U8TH | 6935100\|AF176642.2\|AF176642 Homo sapiens ubiquitin-specific protease ISG43 |
| | | | (ISG43) mRNA, complete cds |
| 347 | AN090263U | U8TH | 12833011\|dbj\|AK002778.1\|AK002778 Mus musculus adult male kidney cDNA, |
| | | | RIKEN full-length enriched |
| 347 | AN090263U | U8TH | 2055430\|U94855.1\|HSU94855 Homo sapiens translation initiation factor 3 47 kDa subunit mRNA, |
| | | | complete cds |
| 354 | AN080732Ua | U8TH | BC006865 Mus musculus, Similar to protein disulfide isomerase-related protein |
| 354 | AN080732Ua | U8TH | 12654930\|gb\|BC001312.1\|BC001312 Homo sapiens, protein disulfide isomerase-related protein |
| 355 | AN080732Ub | U8TH | 12654930\|gb\|BC001312.1\|BC001312 Homo sapiens, protein disulfide isomerase-related |
| | | | protein, clone??? |
| 355 | AN080732Ub | U8TH | 12654930\|BC001312.1\|BC001312 Homo sapiens, protein disulfide isomerase-related |
| | | | protein, clonemRNA, complete |
| 386 | AN082063U | U8TH | AK003052 Mus musculus adult male brain cDNA, RIKEN full-length enriched |
| 386 | AN082063U | U8TH | 665924\|U17248.1\|HSU17248 Human succinate dehydrogenase iron-protein subunit |
| | | | (sdhB) gene, complete cds |
| 489 | ANG2759U | U8TH | 13938085\|gb\|BC007158.1\|BC007158 Mus musculus, procollagen, type I, alpha 2, |
| | | | clone MGC:7369, mRNA, complete |
| 489 | ANG2759U | U8TH | NM_000089.1\| Homo sapiens collagen, type I, alpha 2 (COL1A2), mRNA |
| 508 | ANG2934U | U8TH | 13879335\|gb\|BC006643.1\|BC006643 Mus musculus, clone MGG6582, mRNA, complete cds |
| 508 | ANG2934U | U8TH | 425519\|S65921.1\|S65921 anti-colorectal carcinoma light chain = glycoprotein CANAG-50 |
| 20 | AN010857U | U8TL | 13277680\|BC003745 Mus musculus, Similar to DEAD/H (Asp-Glu-Ala-Asp/His) box polypeptide, |
| | | | complete cds. |
| 20 | AN010857U | U8TL | 2696612\|AB001636.1\|AB001636 Homo sapiens mRNA for ATP-dependent RNA helicase |
| | | | #46, complete cds. |
| 26 | AN010560U | U8TL | 319935\|pir\|\|IJMSCP P-cadherin precursor - mouse |
| 26 | AN010560U | U8TL | 319934\|pir\|\|IJHUCP cadherin 3 precursor - human |
| 50 | AN030134U | U8TL | 55121\|emb\|X63535.1\|MMUFO M.musculus ufo mRNA |
| 50 | AN030134U | U8TL | 238774\|gb\|S65125.1\|S65125 UFO = proto-oncogene [human, NIH3T3 cell, mRNA, 3116 nt] |
| 206 | ANA0360U | U8TL | 13542807\|gb\|BC005605.1\|BC005605 Mus musculus, cyclin D3, clone MGC:5843, mRNA, complete cds |
| 206 | ANA0360U | U8TL | 181246\|M92287.1\|HUMCYCD3A Homo sapiens cyclin D3 (CCND3) mRNA, complete cds |
| 224 | ANA0637U | U8TL | 12963488\|ref\|NM_023118.1\| Mus musculus disabled homolog 2 (Drosophila) (Dab2) |
| 224 | ANA0637U | U8TL | 13111753\|BC003064.1\|BC003064 Homo sapiens, disabled (Drosophila) homolog 2 |
| | | | (mitogen-responsive phosphoprotein), clone MGC:1764 IMAGE:3504380, mRNA, complete cds |
| 292 | AN070836U | U8TL | 6677778\|ref\|NM_009081.1\| Mus musculus ribosomal protein L28 (Rp128), mRNA |
| 292 | AN070836U | U8TL | 13904865\|NM_000991.2\| Homo sapiens ribosomal protein L28 (RPL28), mRNA |
| 208 | ANA0345U | U8TL | 12751446\|gb\|AF335543.1\|AF335543 Mus musculus minor histocompatibility antigen precursor |
| | | | (H47) mRNA |
| 208 | ANA0345U | U8TL | AK026455 Homo sapiens cDNA: FLJ22802 fis, clone KAIA2682 |
| 140 | AN021068D | D1TH | 7949078\|ref\|NP_058034.1\| myosin light chain, phosphorylatable, fast skeletal muscle |
| 140 | AN021068D | D1TH | 14029704\|gb\|AAK52797.1\|AF363061_1 (AF363061) myosin regulatory light chain 2 |
| 375 | AN091863D | D1TH | Partial: 12852119\|dbj\|AK014338.1\|AK014338 Mus musculus 14, 17 days embryo head cDNA, |
| | | | RIKEN full-length |
| 375 | AN091863D | D1TH | 11431721\|ref\|XP_002854.1\| arginine-rich protein [Homo sapiens] |
| 326 | AN051773D | D1TH | 199582\|gb\|AAA39671.1\| (M84367) B(2)-microglobulin [Mus musculus] |
| 326 | AN051773D | D1TH | 4389218\|pdb\|1DDH\|B Chain B, Mhc Class I H-2dd Heavy Chain Complexed With Beta-2 |
| 363 | AN090961D | D1TL | 12841382\|dbj\|BAB25186.1\| (AK007682) putative [Mus musculus] |
| 363 | AN090961D | D1TL | 7021918\|dbj\|BAA91435.1\| (AK000940) unnamed protein product [Homo sapiens] |
| 245 | ANG0934Da | D1TL | Partial: 12858470\|dbj\|AK018655.1\|AK018655 Mus musculus adult male cecum cDNA, |
| | | | RIKEN full-length enriched |
| 245 | ANG0934Da | D1TL | 14249568\|ref\|NP_116236.1\| hypothetical protein FLJ14825 [Homo sapiens] |
| 286 | AN050844D | D1TL | Partial: BC004722 Mus musculus, clone MGC:7901, mRNA |
| 286 | AN050844D | D1TL | Partial: AF130094 Homo sapiens clone FLC0165 mRNA sequence |
| 204 | AN050543DB | D8SH | Partial: 12852985\|dbj\|AK014884.1\|AK014884 Mus musculus adult male testis cDNA, |
| | | | RIKEN full-length enriched |
| 204 | AN050543DB | D8SH | AF139131 Homo sapiens beclin 1 (BECN1) mRNA, complete cds |
| 209 | ANA0334U | U1SL | Partial: 12844078\|dbj\|AK009340.1\|AK009340 Mus musculus adult male tongue cDNA, |
| | | | RIKEN full-length enriched |
| 209 | ANA0334U | U1SL | Partial: BC006176 Homo sapiens, clone IMAGE:4054156, mRNA, partial cds |
| 131 | AN010941U | U1TH | Partial: 12832216\|dbj\|AK002321.1\|AK002321 Mus musculus adult male kidney cDNA, |
| | | | RIKEN full-length enriched |
| 131 | AN010941U | U1TH | NM_005321.1\| Homo sapiens HI histone family, member 4 (H1F4), mRNA |
| 246 | ANG0934Db | U1TH | Partial: 12836025\|dbj\|AK004679.1\|AK004679 Mus musculus adult male lung cDNA, |
| | | | RIKEN full-length enriched. EST: 5333072\|gb\|AI785356.1\|AI785356 uj41a02.x1 |
| | | | Sugano mouse kidney mkia Mus musculus cDNA clone (plus/plus) |
| 246 | ANG0934Db | U1TH | 10140849\|ref\|NP_055107.1\| chromobox homolog 6 [Homo sapiens] |
| 222 | ANG0512U | U1TH | Partial: 2384710\|gb\|AF013969.1\|AF013969 Mus musculus antigen containing |
| | | | epitope to monoclonal antibody MMS-85/12 mRNA, partial cds; Est gb\|AA797801.1\|AA797801 |
| | | | vw33e02.r1 Soares_mammary_gland_NbMMG Mus musculus cDNA clone (used H) |
| 222 | ANG0512U | U1TH | Partial:7243034\|AB037748.1\|AB037748 Homo sapiens mRNA for KIAA1327 protein, partial cds |
| 570 | ANA3226U | U1TL | Partial: 12832641\|dbj\|AK002567.1\|AK002567 Mus musculus adult male kidney |
| | | | cDNA, RIKEN full-length enrichedlibrary, clone:0610011P06, full insert sequence. |
| | | | EST: 10668068\|gb\|BE990041.1\|BE990041 UI-M-BZ1-bfu-c-05-0-UI.s1 NIH_BMAP_MHI2_S1 |
| | | | Mus musculus cDNA clone, (p/p) |
| 570 | ANA3226U | U1TL | BC007034 Homo sapiens, metallothionein 2A, clone MGC: 12397 IMAGE:4051220, |
| 60 | AN030876U | U1TL | Partial: 12841018\|dbj\|AK007456.1\|AK007456 Mus musculus 10 day old male pancreas cDNA |
| 60 | AN030876U | U1TL | BC007799 Homo sapiens, clone IMAGE:4127796, mRNA |
| 197 | AN050727U | U8TH | Partial: 12835429\|dbj\|AK004296.1\|AK004296 Mus musculus 18 days embryo cDNA, |
| | | | RIKEN full-length enriched library |
| 197 | AN050727U | U8TH | AF176642 Homo sapiens ubiquitin-specific protease ISG43 (ISG43) mRNA |
| 504 | | D1SH | 4106365\|gb\|AF068835.1\|AF068835 Mus musculus 54 kDa oligoadenylate |
| | | | synthetase-like protein p540ASL |
| 504 | | D1SH | It is equal to human cDNA NM_003733(AJ225089). It is a 2′-5′-oligoadenylate |
| | | | synthetase-like protein and induced by interferon. |
| 474 | | D1TH | 13386171\|ref\|NM_026835.1\| Mus musculus RIKEN cDNA 1110058E16 gene |
| | | | (1110058E16Rik), mRNA |
| 474 | | D1TH | Member of MS4A(hTM4) family, ligand-gated ion channels. CD20, FcepsilonRIbeta -related. |
| | | | Human homolog MS4A1 gene, 11q13. NM_000139, M89796 |
| 150 | | U1TH | 7305462\|ref\|NM_013654.1\| Mus musculus small inducible cytokine A7 (Scya7), mRNA |
| 150 | | U1TH | The human SCYA7 cytokine (monocyte chemotactic protein 3) cDNA is NM_006273. |
| | | | It is related in inflammation and metastasis. |
| 139 | | U1TH | 12837800\|dbj\|AK005328.1\|AK005328 Mus musculus adult male cerebellum cDNA, |
| | | | RIKEN full-length enriched |
| 139 | | U1TH | Ribosomal L12/L7 C-terminal domain. |

TABLE 2

| | | | | | | | Cell | ECM | | | | | | | |
|---|---|---|---|---|---|---|---|---|---|---|---|---|---|---|---|
| | | | | | | Nuclear | Sur- | (basement | | | | | | Endo- | |
| | | | | | | Regu- | face | membrane | Protein | Protein | Cell | Blood | | thelial | |
| | | | | | TO- | latory | Mar- | and | manu- | degra- | sig- | specific | Muscle | cell | OTH- |
| | 0 | 1 | 8 | 24 | TAL | Factors | kers | factors) | facture | dation | naling | factors | markers | markers | ER |
| U1S | − | + | + | + | 29⁺ | 7 (24%) | 2 | 1 (3%) | 3 (10%) | 5 (17%) | 2 (7%) | 3 (10%) | | | |
| | | | | | | | (7%) | | | | | | | | |
| U8S | − | − | + | + | 11 | 3 (27%) | 1 | 2 (18%) | | | 1 (9%) | 1 (9%) | 1 (9%) | | |
| | | | | | | | (9%) | | | | | | | | |
| D1S | + | − | − | − | 21 | 2 (10%) | | 2 (10%) | 4 (19%) | | 2 (10%) | 2 (10%) | 3 (14%) | | |
| D8S | + | + | − | − | 8 | 2 (25%) | | | 1 (12%) | | | | | 1 (12%) | |
| D1T | + | − | + | + | 39 | 4 (10%) | 2 | 5 (13%) | 3 (8%) | | 4 (10%) | 2 (5%) | 3 (8%) | | |
| | | | | | | | (5%) | | | | | | | | |
| U1T | − | + | − | − | 30 | 7 (23%) | 1 | 3 (10%) | 2 (8%) | | 1 (3%) | | | | |
| | | | | | | | (3%) | | | | | | | | |
| U8T | − | − | + | − | 18 | 4 (22%) | | 3 (17%) | 2 (11%) | 1 (5%) | 1 (5%) | | 2 (11%) | | |
| 0 | | | | | | *8/68 = 12 | 2/ | 7/68 = 10 | 8/68 = 12 | 0/68 = 0 | 6/68 = 9 | 4/68 = 6 | 6/68 = 9 | | |
| | | | | | | | 68 = 3 | | | | | | | | |
| 1 | | | | | | 16/67 = 24 | 3/ | 4/67 = 6 | 6/67 = 9 | 5/67 = 7 | 3/67 = 4 | 3/67 = 4 | 0/67 = 0 | | |
| | | | | | | | 67 = 4 | | | | | | | | |
| 8 | | | | | | 18/97 = 18 | 5/ | 10/97 = 10 | 8/97 = 8 | 6/97 = 6 | 8/97 = 8 | 6/97 = 6 | 6/97 = 6 | | |
| | | | | | | | 97 = 5 | | | | | | | | |
| 24 | | | | | | 14/79 = 18 | 5/ | 8/79 = 10 | 6/79 = 8 | 5/79 = 6 | 7/79 = 9 | 6/79 = 8 | 4/79 = 5 | | |
| | | | | | | | 79 = 6 | | | | | | | | |

⁺Total number of genes differentially-regulated in that stage; not all genes were categorized
*Number of genes expressed in the specific category/total number of genes on and differentially-regulated during that stage

1. A method of determining the angiogenic index of a tissue or cell sample, comprising:
assessing, in a sample, the expression levels of at least two differentially-expressed genes selected from Table 1, whereby said levels are indicative of the angiogenic index.

2. A method of claim 1, wherein assessing is measuring the amounts of mRNA corresponding to said genes present in the sample.

3. A method of claim 2, wherein said measuring is performed by polymerase chain reaction using polynucleotide primers specific for said genes.

4. A method of claim 1, wherein the angiogenic index is assessed by detecting polypeptides coded for by said genes using specific antibodies.

5. A method of claim 1, consisting essentially of assessing expression levels of sustained up-regulated genes or sustained down-regulated genes.

6. A method of claim 1, wherein genes coding for nuclear regulatory factors, cell surface markers, ECM, protein manufacture, protein degradation, cell signaling, and/or endothelial cell markers are assessed.

7. A method of identifying a modulator of a gene, differentially-expressed during angiogenesis, or a polypeptide coded for by said gene, in a cell population capable of forming blood vessels, comprising:
contacting the cell population with a test agent under conditions effective for said test agent to modulate a differentially-expressed gene selected from Table 1, to modulate the biological activity of a polypeptide coded for by said gene, and
determining whether said test agent modulates the gene or polypeptide coded for by it.

8. A method of claim 7, wherein said determining is detecting mRNA or polypeptide of a gene selected from Table 1.

9. A method of claim 7, wherein said determining is detecting the presence or absence of neo-angiogenesis.

10. A method of claim 7, wherein said cell population comprises endothelial cells.

11. A method of regulating angiogenesis in a system comprising cells, comprising:
administering to said system an effective amount of a modulator of a gene, or a polypeptide coded thereby, selected from the differentially-expressed genes of Table 1, under conditions effective for the modulator to modulate said gene or polypeptide, whereby angiogenesis is regulated.

12. A method of claim 11, wherein the modulator is an antibody specific-for said polypeptide.

13. A method of claim 11, wherein the antibody is conjugated to a cytotoxic or cytostatic agent;

14. A method of claim 11, wherein the modulator is an expressible down-regulated gene selected from Table 1.

15. A method of claim 11, wherein regulating angiogenesis is inhibiting angiogenesis;

16. A method of claim 11, wherein regulating angiogenesis is stimulating angiogenesis;

17. A method of claim 11, wherein the system is an in vitro cell culture or in vivo.

18. A method of claim 11, wherein the system is a patent having a cancer, coronary artery disease, myocardial ischemia, or coronary arteriosclerosis.
